(19) United States
(12) Patent Application Publication
(10) Pub. No.: US 2013/0312749 A1
(43) Pub. Date: Nov. 28, 2013

(54) METHOD AND APPARATUS TO ENABLE INHALATION OF AIR OF VARIED TEMPERATURE WITH OR WITHOUT AROMATIC CONDITIONING

(71) Applicant: LifeSense Technologies, LLC, Vashon, WA (US)

(72) Inventors: Robert Bornn, Vashon, WA (US); Laura Worth, Vashon, WA (US); Joshua R. Stewart, Seattle, WA (US)

(21) Appl. No.: 13/900,267

(22) Filed: May 22, 2013

Related U.S. Application Data
(60) Provisional application No. 61/650,279, filed on May 22, 2012.
(60) Provisional application No. 61/734,868, filed on Dec. 7, 2012.

(51) Int. Cl. A61M 16/06; A61M 16/20; A61M 16/10

(52) U.S. Cl. CPC A61M 16/06; A61M 16/1075; A61M 16/208
USPC 128/203.29; 128/206.21; 128/204.15; 128/204.17

(57) ABSTRACT

A device to enable user inhalation and exhalation of air. The air delivered by the device may or may not be aromatically conditioned with user-selected aroma. Further, the air may or may not be thermally conditioned by the device to deliver air that is chilled, warmed, or ambient. The device can include a mask subassembly; an aroma subassembly; a neck portion; a base subassembly with thermal mass; and a soft, insulated cover assembly. Methods of using the device i) teach breathing techniques for relaxation using sensory feedback from the device; ii) educate, establish and heighten awareness of positive sleep routines that are considered important in healthy sleep hygiene; iii) aid in identifying behaviors that are not conducive to good sleep hygiene; iv) assist the user in identifying dysfunctional rumination and belief systems about sleep and relaxation; and v) educate the user about breathing techniques and relaxation.

### BACKGROUND

1. Technical Field

The present disclosure relates to devices and methods for accessing the psychophysiological, potentially healthful effects of daytime and nighttime relaxation, including reducing sleep latency (the time it takes to fall asleep) and improving the maintenance of healthy sleep. The present disclosure further relates to devices and methods for accessing mechanisms of alertness and wakefulness.

2. Description of the Related Art

For millions of people every night, easily falling asleep or staying asleep throughout the night is a formidable challenge. When chronic insomnia reduces the total time asleep, healthy and restorative sleep is reduced. The consequence of insomnia is increased physiological and emotional stress, cognitive decline, and further disordered sleep. It has been reported that severe, chronic insomnia is experienced by more than 30 million Americans of all ages. Inadequate duration and quality of sleep are associated with reduced “healthspan,” longevity, and well-being during waking hours.

In an aging population with more chronic disease, more sleep disturbances (including secondary insomnia), are noted. This poor sleep quality is especially likely in elders to exacerbate stress, cognitive decline, and result in yet more sleep problems—which in turn contribute to insomnia. Risk for elders of cardiovascular disease and stroke; diabetes; and decline in cognitive, immune, and other functions (including sexual dysfunction) are all likely to increase when sleep duration and quality are inadequate.

Pharmaceutical interventions are contraindicated for chronic insomnia. Their effectiveness, if any, is short-lived in that tolerance occurs within a few weeks. Sleeping pills can reduce time spent in restorative, deep sleep and are sometimes accompanied by side effects like hallucinations or frightening sleepwalking episodes combined with dangerous daytime drowsiness. A recently published research study has demonstrated a strong association between sleeping pills and cancer.

Many other clinical and self-help intervention strategies are typically used with varying degrees of success.

It has been documented that insomnia and poor sleep quality are negatively associated with “healthspan,” longevity, and well-being for all generations. Insomnia may be classified by severity, type, or cause. Regardless of a patient's age or the severity, type, or cause of a person's insomnia, the present invention provides relief and comfort because it aids in relaxation, which is a helpful precondition to sleep. Further, use of the present invention incorporates measures to improve sleep hygiene with longer-lasting positive lifestyle modifications.

Adult Insomniacs: 58% of American adults have experienced insomnia at least a few nights a week, according to the National Sleep Foundation's 2002 poll. In April 2012, this 58% represented approximately 136 million adults (U.S.).

Young to Middle Age Adults: Among adult Americans younger than 55, 62% report experiencing insomnia. Daytime and nighttime stress and “over-thinking brains” are connected with insomnia in these younger adults. Their insomnia is likely to be “primary insomnia” (no other underlying medical conditions).

Elder Adults: Among adult Americans between 55 and 84, 45% report insomnia. Their insomnia is more likely to be “secondary insomnia” (connected to other underlying medical conditions). The elder demographic is expected to continue growing rapidly along with the increase in this population's chronic disease and sleep disturbances. In turn, this poor sleep quality is likely to exacerbate stress, cognitive decline, inflammation, pain and other sleep disorders. Risk for elders of cardiovascular disease and stroke; diabetes; and decline in cognitive, immune, and other functions (including sexual dysfunction) are all likely to increase when sleep duration and quality is inadequate.

Severity: Insomnias can be transient (less than a week), acute (less than a month), and chronic (longer than a month).

Types: Insomnia may occur prior to sleep (sleep onset insomnia) or it may be associated with awakenings during the night with difficulty falling back to sleep (nocturnal awakenings).

Cause: “Primary Insomnias” are those without connection to other underlying medical conditions. Widespread “secondary Insomnias” are connected to medical conditions such as excessive nighttime urination, pain disorders, stress, and illness, all of which may increase with age.

Devices, pharmaceuticals, supplements and botanicals, aromatic essential oils or flowers for aromatherapy, sleep hygiene educational programs, and methods for relaxation and improved sleep health are currently marketed for self-care, or offered by those who are tasked professionally with managing stress and insomnia in their clients or patients. Such practitioners include medical professionals and complementary and alternative medicine (CAM) practitioners.

Examples of devices include scented and unscented eye masks to cover the eyes for darkness during sleep and to induce relaxation that are marketed to facilitate relaxation and sleep.

Methods, devices, and systems have been developed by Dr. Eric Nofzinger to apply cooling therapy to the skin of the forehead to cool the brain's prefrontal cortex prior to and/or during sleep. In his clinical research, cooling the prefrontal cortex has been reported by Dr. Nofzinger to be helpful in inducing sleep for insomniacs. Dr. Nofzinger reports that his research using fMRIs supports the efficacy of hypothermic treatment for insomnia. This approach may be costly and uncomfortable for the user (see, for example, U.S. Patent Application Publication No. 2011/0125238 A1).

With respect to pharmaceuticals, although they are easy for patients to request and for primary care physicians to prescribe, many sleep specialists recommend against the use of pharmaceuticals to treat chronic insomnia. Some of the initial effectiveness of sleeping pills is based on the placebo effect. After a few weeks, effectiveness is reduced due to tolerance and the problem of chronic insomnia is inadvertently intensified. As a long-term solution, sleeping pills can have a negative effect on health, especially for elders, in that they can reduce time spent in restorative, deep sleep necessary for health and repair of daily cellular damage. They can also be accompanied by serious side effects like hallucinations, frightening sleepwalking episodes, and dangerous daytime drowsiness.

Recent scientific data analysis shows a strong correlation between sleeping pills and cancer. The analyzed data was originally gathered from 40,000 people during a 5 year period by the American Cancer Society when it conclusively demonstrated the link between cigarettes and cancer. Analysis of the ACS data by Scripps Health shows that as few as 18 sleeping pills per year increase cancer risk fourfold. Taking a sleeping pill each night has been said by a principle investigator of the study to comparatively be approximately the equivalent risk factor of smoking a pack of cigarettes per day. The sleeping pills in question were common hypnotics such as Ambien and Valium. Whether this effect is causal or associated with pre-existing sleep disorders resulting in higher mortality rates has not been determined.

The use of supplements and botanicals is also known. Various hormones and botanicals are reputed to aid in managing insomnia. They include the hormone melatonin, botanicals like the chamomile flower, and herbs like lemon balm and valerian extract. Because they are marketed as health supplements, these hormones, botanicals, and herbs are not reviewed by the Food and Drug Administration. There are no regulatory controls on effectiveness, quality, purity, or dosage. For example, MIT research in 2001 showed that with melatonin use, the most effective dose of melatonin is 0.3 mg before sleep. However, the common dose of a melatonin supplement sold in health food stores is 3.0 mg (which may actually limit effectiveness). Some brands suggest 20 mg if directed by a “healthcare practitioner,” which may be a dangerous dose.

Aromatherapy using various phytochemicals is recommended by many complementary and alternative medicine and wellness practitioners. Phytochemicals and essential oils are readily available as self-care modalities. They are relied upon by many as a complementary or alternative medicine to aid in achieving relaxation and reducing insomnia. The National Institutes of Health has sponsored research into the efficacy of aromatherapy for various applications. For example, certain phytochemicals appear to reduce agitation associated with Alzheimer's syndrome without the dangers that can be associated with the use of antipsychotics in that population.

There is a plethora of consumer self-care products marketed to provide aromas for relaxation and improved sleep. For example, one such series of devices functions as a scented eye mask to cover the eyes for darkness during sleep.

Cognitive-Behavioral Therapy for Insomnia (CBT) is also known. Consumer services and programs are currently marketed to the public to encourage health and wellness, including cognitive behavioral therapy programs to encourage relaxation and healthy sleep onset and maintenance. Such CBT insomnia programs are challenging to achieve, requiring long-term commitment.

Foundational to such CBT programs and services is the work of cardiologist, Dr. Herbert Benson, who pioneered scientific investigation at Harvard Medical School into certain health-promoting physiological responses to deep breathing relaxation techniques associated with meditation. In his research Dr. Benson established the relationship of breathing techniques to the physiology of relaxation, which he described as the body's compensating response to its opposite “fight or flight” response to stressful situations. In addition to extensive peer-reviewed literature published during his decades-long career of investigating this response, he published a self-care book, Relaxation Response (1975 and 2000). Dr. Benson identified the positive clinical physiological changes that occur with meditative breathing, such as clinically lowered blood pressure and lowered blood levels of cortisol and epinephrine, which in excess can be associated with stress.

An example of a CBT insomnia program was developed at Harvard Medical School under the leadership of Dr. Gregg D. Jacobs. He describes it in his book, Say Goodnight to Insomnia: the Only Natural Treatment Scientifically Proven to Conquer Insomnia (1998). The Harvard program encourages insomnia patients to achieve the health-promoting “Relaxation Response” identified by Dr. Benson.

Currently Dr. Jacobs markets a 5-week program of classes and products for relaxation and insomnia through various websites.

Some CBT programs encourage cognitive adjustments to the way patients think about their sleep. These changes are encouraged by educating patients about the nature of sleep and helping them minimize fear, dread, and anxiety. CBT patients are encouraged to stop catastrophizing about insomnia. They can achieve more hopeful assessments of their prognosis and a more realistic understanding of the actual effects of their insomnia, which is thought to help break its hold over the patient.

Recommended cognitive corrections encourage insomniacs to intensively explore new ways of thinking about the mastery of sleep, based on currently established medical knowledge.

CBT programs encourage behavioral lifestyle corrections designed to optimize environments and behavioral habits to improve physiology for healthy sleep. The behavioral corrections recommended by such programs may be facilitated by check lists, a well-established behavioral modification technique. Such program suggestions may, for example, include establishing regular times to go to bed and rise each day, changes in the sleep environment, dietary improvements, smoking cessation, and creation of before-bed rituals. These programs may encourage the practice of daytime and nighttime periods of physiologic relaxation to reduce the stress-related excessive levels of epinephrine and cortisol in the blood.

Mindfulness practices like mindfulness-based stress reduction (MBSR) hale from an ancient Buddhist tradition. MBSR is the subject of hundreds of scientific studies funded by NIH, The National Center of Complementary and Alternative Medicine.

For example, beginning in 1979, Dr. Jon Kabat-Zinn began to study this approach to stress reduction at Massachusetts Medical Center. The work coalesced in publishing results of his work in the self-care book, Full Catastrophe Living: Using the Wisdom of Your Body and Mind to Face Stress, Pain, and Illness (1990). Mindfulness meditation is a term he uses for practices and techniques he employs in clinical groups and with individual patients to systematically achieve MBSR. In the West his clinical program and his scientific research into meditation and mindfulness-based stress reduction have helped to popularize many ancient traditions going back many millennia.

MBSR has been clinically proven to aid in managing pain, anxiety, and physical and emotional stressors that may inhibit sleep onset.

Self-hypnosis is another known technique. Hypnotic techniques can also be used to reduce thought patterns connected with insomnia or stress. They can also be used to induce behaviors that are conducive to relaxation and managing insomnia. For example, they have been shown to aid in smoking cessation related to sleep hygiene and insomnia, changing eating and drinking habits, and reducing pain that may contribute to nighttime stress and sleeplessness. Hypnotic techniques may be used separately or in connection with CBT.

Sleep Hygiene Programs are also known. Recommendations designed to improve sleep hygiene are widely offered by physicians and other health and wellness professionals. Sleep hygiene advice also abounds in self-help books and magazines that advise the public on how to achieve healthier sleep. Most sleep hygiene advice involves self-care change in thoughts and beliefs associated with nighttime stress and lifestyle modifications to diet and behavioral modification, often requiring changes in longstanding habits.

### BRIEF SUMMARY

It has been recognized that the devices and methods of the present disclosure can help induce daytime and nighttime relaxation. The devices and methods of the present disclosure may also induce certain physiological responses associated with relaxation and calmness that may counter the harm of stress on the human body. The devices and methods of the present disclosure may further harmlessly enable sleep, either at sleep onset or following nighttime arousals. Additionally, devices and methods of the present disclosure may harmlessly enable wakeful or alert states during the day or following sleep.

The present disclosure relates to a plurality of devices and a plurality of methods for self-care or for practitioner-guided administration to induce relaxation, sleep, wakefulness, or alertness.

In one aspect, the devices disclosed herein may permit user-selection of a plurality of combinations of aromatic air and/or air temperature: (for example, i. cool air with aroma, ii. cool air without aroma, iii. warm air with aroma, iv. warm air without aroma, v. ambient air with aroma, vi. ambient air without aroma).

In another aspect, the plurality of devices disclosed herein may permit user-selection of sensory feedback about personal breathing techniques based on combinations of aromatic air and/or air temperature, and/or air flow. For example, a combination of aroma, chilled air, and air flow sensations can make the user more aware of their depth, duration, steadiness, rate, and rhythmicity of inhalation and exhalation.

Another aspect includes user-selected methods for sensory feedback training in established breathing techniques to induce relaxation, alertness, and/or sleep.

A further aspect includes user-selected methods incorporating self-care or professionally administered CBT principles of cognitive and behavioral modification related to inducing relaxation, alertness, and/or sleep.

In another aspect, a comprehensive approach to relaxation, alertness, and insomnia, consisting of a device and methods, which may be more effective for some individuals than any of the isolated approaches represented by conventional approaches.

In one aspect, several subassemblies are integrated in one device. In other embodiments, options are based on user-selected subassemblies, size, shape, and accessory configurations.

The devices disclosed herein may be combined by the user with various methods disclosed herein and options according to a desired effect.

In one aspect, the effectiveness of the devices and methods of the present disclosure is based in part on device and method-based factors including brain temperature changes, aromatically conditioned air, and/or cognitive behavioral intervention.

With respect to brain temperature changes, the devices disclosed herein may make use of the relationship of brain temperature changes to wakefulness and sleep, including the relationship of insomnia to hypothermic treatments that cool the brain. It has been recently demonstrated, using fMRIs to detect brain activity, that cooling the brain's prefrontal cortex appears to reduce sleep latency. Similarly, increasing brain temperature appears to be associated with waking from sleep.

With respect to aromatically conditioned air, the devices disclosed herein may utilize a self-care modality, using naturally occurring phytochemical aromas to manage stress and promote relaxation. Relaxation and sleep-onset are encouraged by inhaling specially-targeted, natural, aromatic phytochemicals using the devices and a uniquely simple, learned-breathing procedure, optimally described as inhalation through the nose and exhalation through the mouth. The devices deliver user-selected aromas (such as vanilla and lavender) that may be conducive to reduced agitation, facilitating conditions for timely sleep-onset and maintenance of restorative and healthy sleep to improve a person's well-being.

With respect to cognitive behavioral intervention, the novel devices and methods disclosed herein may work as a whole system to synergistically utilize self-care or professionally administered contemporary principles of Cognitive Behavioral Therapy (CBT) for improved sleep hygiene, biofeedback principles related to relaxation and sleep onset, mindfulness breathing, and deep breathing techniques. CBT principles have been shown to enhance the body's physiological response to relaxation. These physiological responses to relaxation are clinically shown to reduce insomnia and improve sleep quality. For example, reduction of excessive blood cortisol and epinephrine levels through relaxation, whether during the day or night, is shown to improve sleep quality and reduce insomnia.

Embodiments of the devices disclosed herein can include five primary subassemblies: (i) a mask subassembly designed to fit securely over the nose or mouth and be held gently in place with voluntary effort by the user; (ii) an aroma subassembly designed to optionally enable inhalation of aromatic air with voluntary effort by the user, (iii) a neck portion, (iv) a base subassembly designed to enable voluntary inhalation of air that is chilled, heated, or ambient temperature for varied time periods, and (v) a cover subassembly designed to act as a filter for inhaled air to and maintain optimal temperature and ensure comfortable use.

Embodiments of the devices disclosed herein may deliver user-selected chilled, warmed, and/or ambient-temperature air nasally or orally to assist in varied temperature ranges that tend to encourage relaxation, alertness, or to enable sleep.

Further, embodiments of the devices disclosed herein may deliver aromatically conditioned air and phytochemical terpenes nasally or orally to the nasopharynx, olfactory tract, and the olfactory bulb of the brain.

Further, embodiments of the devices disclosed herein may provide aural air movement sounds that result from inhaled and exhaled air moving through the nose, through the mouth, and/or through the mask subassembly and more specifically through valve subassemblies.

Further, the devices disclosed herein may provide temperature and/or aromatic and/or aural sensory feedback to the user of the depth, steadiness, rate and rhythmicity of inhalations and/or exhalations.

Embodiments of the present invention can include a novel method of using the device means wherein voluntary or assisted inhalation of air is accomplished through the nose.

In other aspects, the present disclosure includes a method of using the devices disclosed herein in which voluntary or assisted exhalation of air is accomplished through the mouth.

Further, other aspects include a method of using the devices disclosed herein in which voluntary or assisted inhalation of air through the nose and voluntary or assisted exhalation of air through the mouth enables sensory feedback to the user.

Further, user-selected methods for sensory feedback training may be combined by the user with a plurality of user-selected devices disclosed herein to provide sensory feedback to the user from: (i) aromatic air; and/or (ii) air temperature; and/or (iii) sounds generated by the device means from inhalation and exhalation. These methods for sensory feedback training may enable relaxation, sleep, and/or alertness.

Further, user-selected methods disclosed herein for cognitive and behavioral modification may be combined by the user with one or more of the devices disclosed herein synergistically for amplified positive effect. By way of example, but not limitation, such methods may include relaxation or sleep log formats, journal formats and check lists that are behaviorally anchored in use of the device that help to establish healthy sleep routines, educate the user about good sleep hygiene habits, or encourage awareness of sleep-limiting rumination patterns or negative thoughts and belief systems about sleep. The methods used in combination with the devices for cognitive and behavioral modification may enable relaxation, sleep, and/or alertness.

In another aspect, an integrated assembly is provided. The integrated assembly combines a mask subassembly, an aroma subassembly, and a base subassembly. Based on user-selection of temperature, aroma, and a plurality of self-care or professionally administered sensory feedback techniques and cognitive behavioral modification techniques, the integrated assembly may enable daytime and nighttime relaxation, alertness, and/or sleep.

The integrated subassembly may include a user-selected, self-administered combination of (i) chilled air to the brain through nasal and/or oral inhalations; and/or (ii) air that is aromatically conditioned with active phytochemicals to the olfactory tract through user-selected nasal and/or oral inhalations; and/or (iii) sounds, such as air movement sounds, that result from inhaled and exhaled air moving through the nose, through the mouth, and/or through one-way valves; and/or air flow sensations. This configuration appears to slightly cool the olfactory tract and nasopharynx and, by inference, the proximal brain mass, which may be helpful in inducing calmness, relaxation, and sleep. This configuration also delivers phytochemical terpenes to the olfactory bulb of the brain from user-selected, aromatic essential oils or flowers that may induce calmness, relaxation, or alertness. This configuration also delivers sensory feedback for use in methods described herein.

In another aspect, a method of using the integrated assembly includes voluntary or assisted inhalation of air through the nose and voluntary or assisted exhalation of air through the mouth enables sensory feedback to the user.

Other aspects include methods utilizing sensory feedback to the user from the integrated subassembly of the duration, depth, steadiness, rate and rhythmicity of the user's inhalation and exhalation to induce relaxation and/or sleep onset and/or alertness. Other aspects include methods including cognitive and behavioral modification techniques that may enable relaxation and/or sleep onset and/or alertness.

In another aspect, the integrated assembly may be used to enable alertness. In this aspect, the user-selected temperature of the base module may be ambient temperature or warmed above ambient and the user-selected aroma is compatible with inducing wakefulness or alertness. For example, coffee aromas may be associated with wakefulness.

In another embodiment, a mask subassembly is combined with a base subassembly to enable inhalation, at the user's option, of chilled air to the brain through user-enabled nasal and/or oral inhalations. When applied in combination with at least one of the methods disclosed herein, the chilled temperature of the air alone provides direct sensory feedback to the user of the depth, duration, steadiness, rate and/or rhythmicity of their respiration to assist in learning breathing techniques that induce calmness, relaxation, and sleep onset. Further, this method may deliver cooled air to the brain from the base subassembly through user-enabled nasal and/or oral inhalations that may enable alertness, relaxation, or calmness.

In another embodiment, a device combines a mask subassembly with a base subassembly and delivers, at the user's option, warmed air to the brain through user-enabled nasal and/or oral inhalations. When applied in combination with at least one of the methods disclosed herein, the warmed temperature of the air provides direct sensory feedback to the user of the depth, duration, steadiness, rate, and/or rhythmicity of their respiration to assist in learning breathing techniques that enable relaxation and sleep onset or may enable alertness, relaxation, or calmness.

In another embodiment, a device includes a mask subassembly and an aroma subassembly. In this embodiment, the aroma subassembly contains self-selected aromatic oils or flowers that, through user-enabled nasal and/or oral inhalations, at the user's option, can introduce aromatically conditioned air at ambient temperature through the mask subassembly that may enable alertness, relaxation, or calmness. When applied in combination with at least one of the methods disclosed herein, the aromatically conditioned air provides direct sensory feedback to the user of the depth, duration, steadiness, rate and/or rhythmicity of their respiration to assist in learning breathing techniques that may enable relaxation and sleep onset or may induce alertness, relaxation, or calmness.

Another embodiment permits a user-selected combination of a mask subassembly, an aroma subassembly, and a base subassembly and delivers, through user-enabled nasal and/or oral inhalations, at the user's option: chilled, aromatically conditioned air to the olfactory tract through nasal and/or oral inhalations. When applied in combination with at least one of the methods disclosed herein, the chilled, aromatically conditioned air provides direct sensory feedback to the user of the depth, duration, steadiness, rate and/or rhythmicity of their respiration to assist in learning breathing techniques that may enable relaxation and sleep onset. Further, this method delivers phytochemical terpenes to the olfactory bulb of the brain from user-selected essential oils or flowers that may induce alertness, relaxation, or calmness.

In another embodiment, a user-selected combination of a mask subassembly, an aroma subassembly, and a base subassembly delivers, through user-enabled nasal and/or oral inhalations, at the user's option: i) warmed air to the brain through nasal and/or oral inhalations; and/or ii) aromatically conditioned air to the olfactory tract through user-selected nasal and/or oral inhalations. When applied in combination with at least one of the methods disclosed herein, the warmed and aromatically conditioned air provides sensory feedback to the user of the depth, duration, steadiness, rate and/or rhythmicity of their respiration to assist in learning breathing techniques that enable relaxation and sleep onset. Further, this method delivers phytochemical terpenes to the brain from user-selected essential oils or flowers.

In another aspect, the devices of the present disclosure may provide a plurality of user-selected combinations of the subassemblies. For example, further embodiments can include a mask subassembly alone for user-selected applications in which aural sensory feedback alone is desirable or applications wherein no sensory feedback is desirable. For example, the novel mask interface could be used in other respiratory devices such as oxygen masks and continuous positive airway pressure (CPAP) devices.

A further embodiment can include the mask subassembly and the aroma subassembly for aural and aromatic sensory feedback.

Further, the devices of the present disclosure may provide a plurality of user-selected combinations of the subassemblies for additional applications. For example, the mask subassembly may be used in further applications wherein the present invention's novel interface with the human face is desirable.

Further, a smaller embodiment for purse, briefcase, or desk can be used away from home for relaxation or to facilitate napping.

Further, product accessories may enhance the efficacy of the device means. For example, a selection of various sizes of insulated carrying cases means will maintain the device at a range of desired temperatures.

In one aspect, a method of using the device includes voluntary or assisted inhalation of air through the nose that enables inhalation of air at a user-selected temperature range, thereby delivering air through the nasopharynx, olfactory tract and, by inference, chilling or warming the olfactory bulb and proximal brain mass in the prefrontal cortex.

In another aspect, a method of using a mask subassembly device includes voluntary or assisted exhalation of air through the mouth from the lungs after it has been warmed by body heat.

In another aspect, a method of using a mask subassembly device includes voluntary or assisted exhalation of air accomplished through the nose from the lungs after it has been warmed by body heat.

In another aspect, a method includes enabling voluntary inhalation of air at a user-selected temperature range to also support the voluntary or assisted inhalation of aromatically conditioned air through the nose and exhalation of aromatically conditioned air through the mouth or nose.

In another aspect, a method includes using at least one of the devices disclosed herein to enable voluntary or assisted inhalation of air through the nose and voluntary or assisted exhalation of air through the mouth or nose to enable sensory feedback to the user.

In another aspect, a method for sensory feedback may be combined by the user with at least one of the devices disclosed herein. The sensory feedback may provide training to the user based on i) air temperature and/or ii) aromatic air and/or iii) sound generated by the user from inhalation and exhalation; and iv) air flow sensations, all four sources of sensory feedback being created in connection with use of the device means. The method may enable relaxation, sleep, and/or alertness

In one aspect, immediate sensory feedback of the user's breathing rate, duration, depth, steadiness, and rhythmicity is provided to the user by the sensation of cold, aromatic air moving through nasal passages and the back of the throat and mouth. In another aspect, immediate sensory feedback of the user's breathing rate, duration, depth, steadiness, and rhythmicity is provided by aural means such as air movement sound that results from air moving through the nose, through the mouth, and/or through one-way valves in the mask subassembly.

In another aspect, immediate sensory feedback using the user's breathing rate, duration, depth, steadiness, and rhythmicity is provided by air flow sensations.

In another aspect, immediate temperature and aromatic sensory feedback of the user's breathing rate, duration, depth, steadiness, and rhythmicity is provided to assist the user in learning a plurality of user-selected breathing techniques to induce relaxation, sleep, or alertness such as deep breathing, mindful breathing, mindfulness meditation, guided meditation, self-hypnosis, visualization and/or sensory recall. Once they are learned, these techniques can be used without the present invention to induce relaxation, sleep, or alertness.

A user-selected plurality of methods for cognitive and behavioral modification may be combined by the user with at least one of the devices disclosed herein to induce relaxation, sleep, and/or alertness. Such plurality of methods for cognitive and behavioral modification may incorporate self-administered or professionally administered instructions and/or forms for written exercises and reference use of the devices. Methods engendering positive behavioral changes may lead these behaviors to become anchored by use of the devices. Such methods would be used specifically in connection with the plurality of novel device means of the present invention. Such methods can include by way of example but not limitation: i) journal forms, ii) check lists, iii) calendars, and/or iv) schedules.

### BRIEF DESCRIPTION OF THE SEVERAL VIEWS OF THE DRAWINGS

In the drawings, identical reference numbers identify similar elements or acts.

FIG. 1 is an isometric view of an integrated assembly according to one aspect of the present disclosure.

FIG. 2 is an exploded, isometric view of the integrated assembly of FIG. 1.

FIG. 3a is a top view of the integrated assembly of FIG. 1.

FIG. 3b is a side view of the integrated assembly of FIG. 1.

FIG. 4a is a pictorial view of the integrated assembly of FIG. 1.

FIG. 4b is a front view of the integrated assembly of FIG. 1.

FIG. 5a is a pictorial view of the integrated assembly of FIG. 1.

FIG. 5b is a pictorial view of the integrated assembly of FIG. 1.

FIG. 6a is a cross-sectional view of the integrated assembly of FIG. 1 taken along line 6-6 in FIG. 3a.

FIG. 6b is partial cross-sectional view of the integrated assembly of FIG. 1 with the neck portion bent upwards.

FIG. 6c is partial cross-sectional view of the integrated assembly of FIG. 1 with the neck portion bent downwards.

FIG. 7 is a cross-sectional view of the integrated assembly of FIG. 1 taken along line 7-7 in FIG. 3b.

FIG. 8 is an isometric view of the integrated assembly of FIG. 1 with a thermal cover.

FIG. 9a is a top view of the integrated assembly of FIG. 8.

FIG. 9b is a side view of the integrated assembly of FIG. 8.

FIG. 10 is a cross-sectional view of the integrated assembly of FIG. 8 taken along line 10-10 in FIG. 9a.

FIGS. 11a-11f are various views of the mask portion of the integrated assembly of FIG. 1.

FIG. 12 is an isometric view of an integrated assembly according to another aspect of the present disclosure.

FIG. 13 is an exploded, isometric view of the integrated assembly of FIG. 12.

FIG. 14a is a top view of the integrated assembly of FIG. 12.

FIG. 14b is a side view of the integrated assembly of FIG. 12.

FIG. 15a is a pictorial view of the integrated assembly of FIG. 12.

FIG. 15b is a front view of the integrated assembly of FIG. 12.

FIG. 16a is a pictorial view of the integrated assembly of FIG. 12.

FIG. 16b is a pictorial view of the integrated assembly of FIG. 12.

FIG. 17a is a cross-sectional view of the integrated assembly of FIG. 12 taken along line 17-17 in FIG. 14a.

FIG. 17b is partial cross-sectional view of the integrated assembly of FIG. 12 with the neck portion bent upwards.

FIG. 17c is partial cross-sectional view of the integrated assembly of FIG. 12 with the neck portion bent downwards.

FIG. 18 is a cross-sectional view of the integrated assembly of FIG. 8 taken along line 18-18 in FIG. 14b.

FIG. 19 is an isometric view of the integrated assembly of FIG. 12 with a thermal cover.

FIG. 20a is a top view of the integrated assembly of FIG. 19.

FIG. 20b is a side view of the integrated assembly of FIG. 19.

FIG. 21 is a cross-sectional view of the integrated assembly of FIG. 19 taken along line 21-21 in FIG. 20a.

FIGS. 22a-22f are various views of the mask portion of the integrated assembly of FIG. 12.

FIG. 23a is an exploded side view of an aroma subassembly according to one aspect.

FIG. 23b is an exploded isometric view of the aroma subassembly of FIG. 23a.

FIG. 23c is a front view of the aroma subassembly of FIG. 23a in an assembled state.

FIG. 23d is a side view of the aroma subassembly of FIG. 23a in an assembled state.

FIG. 23e is a cross-sectional view of the aroma subassembly of FIG. 23a in an assembled state.

FIG. 24a is a top view of an integrated assembly according to another aspect of the present disclosure.

FIG. 24b is a pictorial view of the integrated assembly of FIG. 24a.

FIG. 24c is another pictorial view of the integrated assembly of FIG. 24a.

FIG. 24d is a side view of the integrated assembly of FIG. 24a.

FIG. 24e is a front view of the integrated assembly of FIG. 24a.

FIG. 24f is a cross-sectional view of the integrated assembly of FIG. 24a.

FIG. 25a is a side view of an integrated assembly according to another aspect of the present disclosure.

FIG. 25b is a pictorial view of the integrated assembly of FIG. 25a.

FIG. 25c is a top view of the integrated assembly of FIG. 25a.

FIG. 25d is another pictorial view of the integrated assembly of FIG. 25a.

### DETAILED DESCRIPTION

In the following description, certain specific details are set forth in order to provide a thorough understanding of various embodiments of the invention. However, one skilled in the art will understand that the invention may be practiced without these details.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its broadest sense, that is as meaning “and/or” unless the content clearly dictates otherwise.

The Abstract of the Disclosure provided herein is for convenience only and does not interpret the scope or meaning of the embodiments.

In one example, a device of the present disclosure includes several subassemblies that may be combined into one integrated assembly. The shape and exterior texture of the integrated assembly permits enhanced effectiveness of the device and the methods disclosed herein by permitting comfortable and easy “cradling” of the integrated assembly.

The integrated assembly can include four subassemblies: (i) a mask subassembly designed to fit securely over the nose or mouth and be held in place with voluntary effort by the user; (ii) an aroma subassembly designed to optionally enable inhalation of aromatic air, with voluntary effort by the user; (iii) a base subassembly designed to enable voluntary inhalation of air that is chilled, heated, or ambient temperature for varied time periods; and (iv) a thermal cover subassembly designed to act as a filter for inhaled air and maintain optimal temperature.

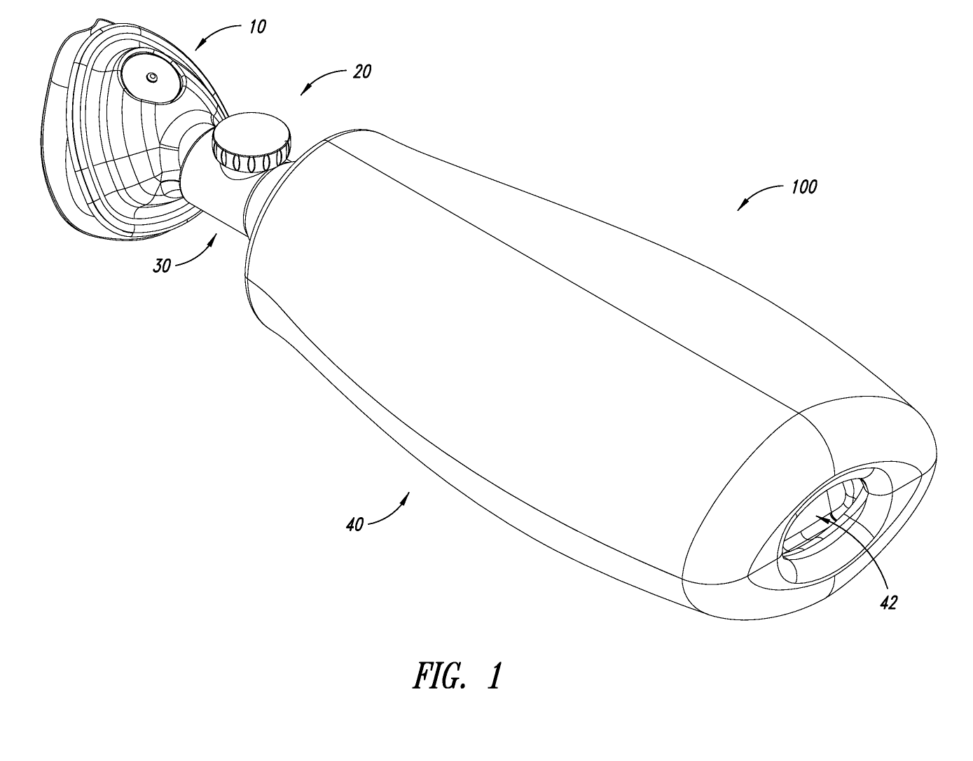

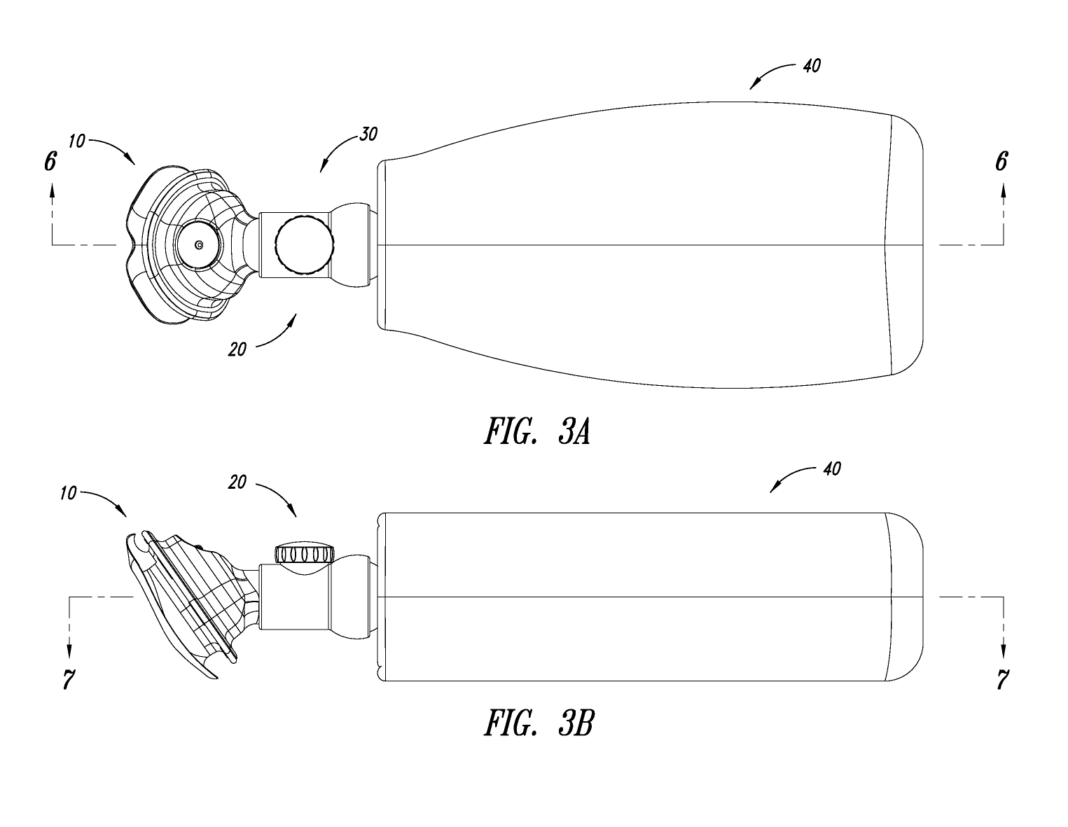

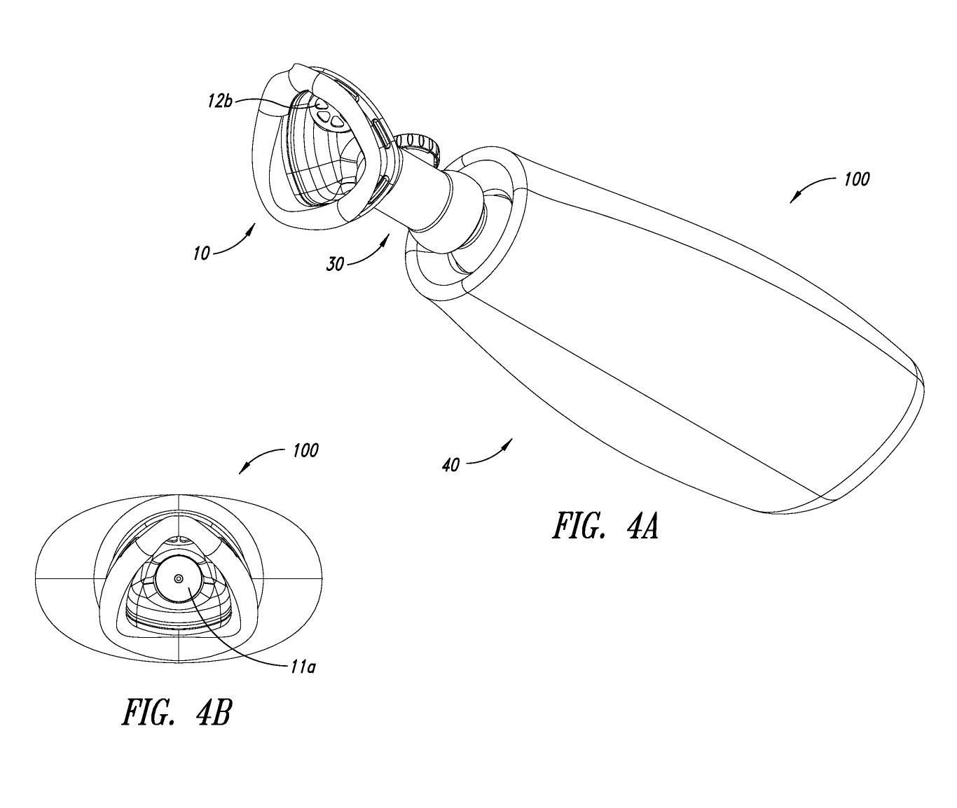

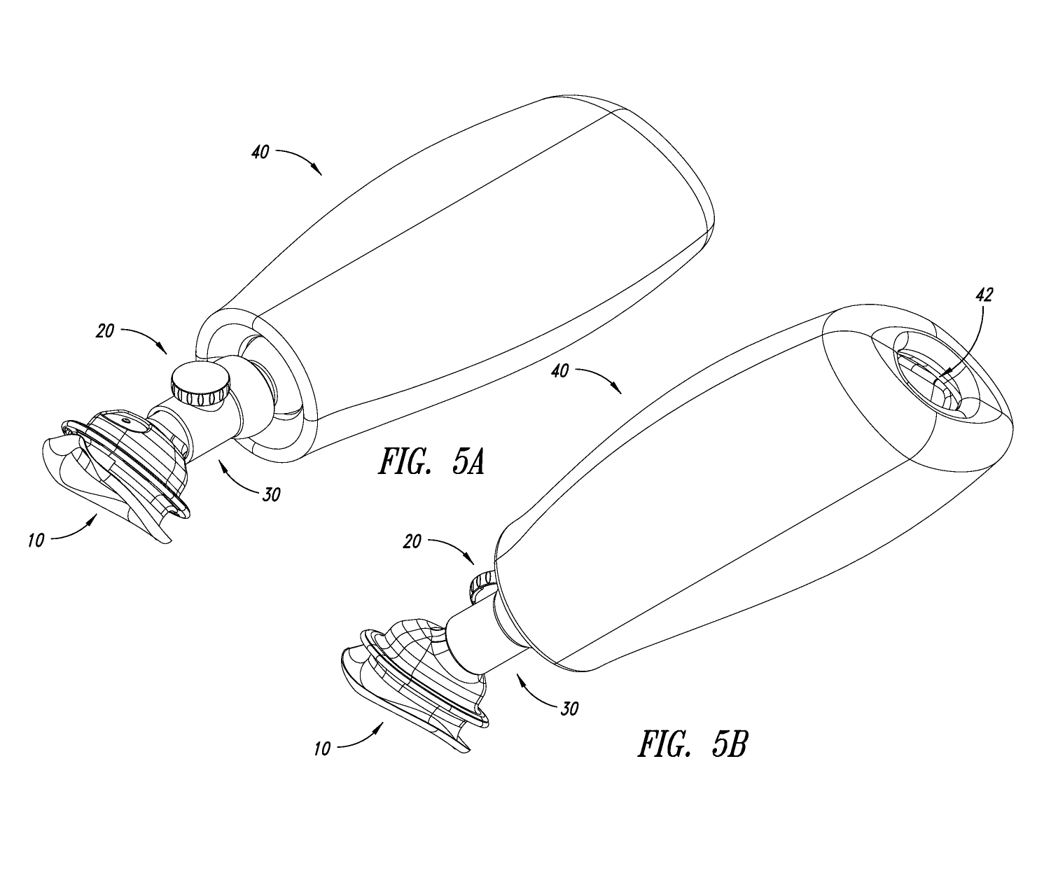

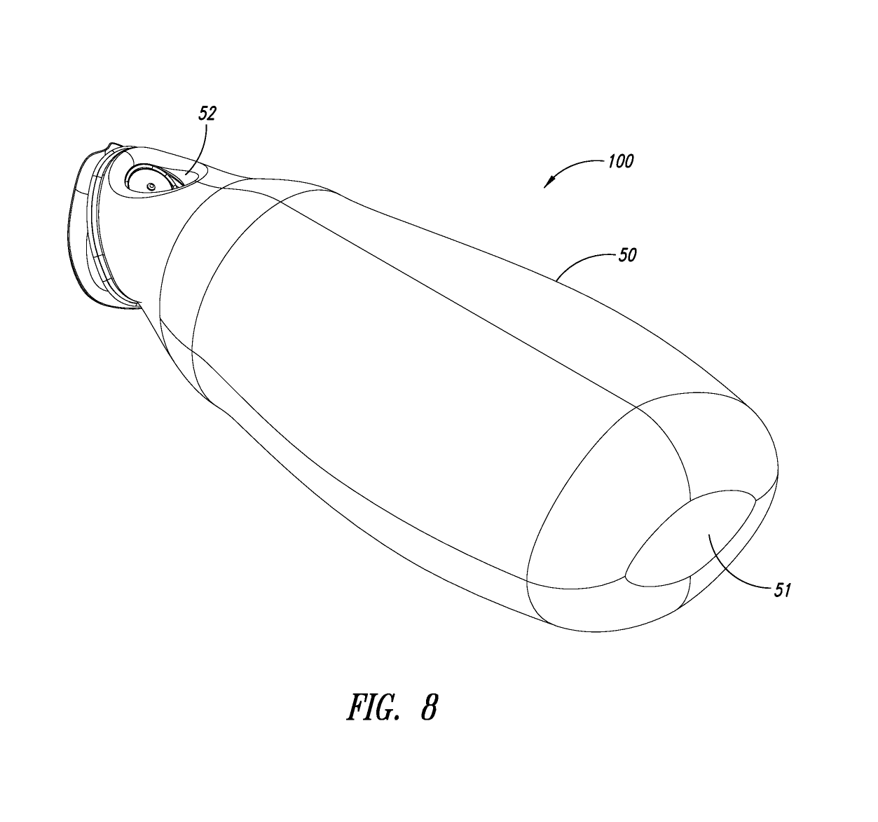

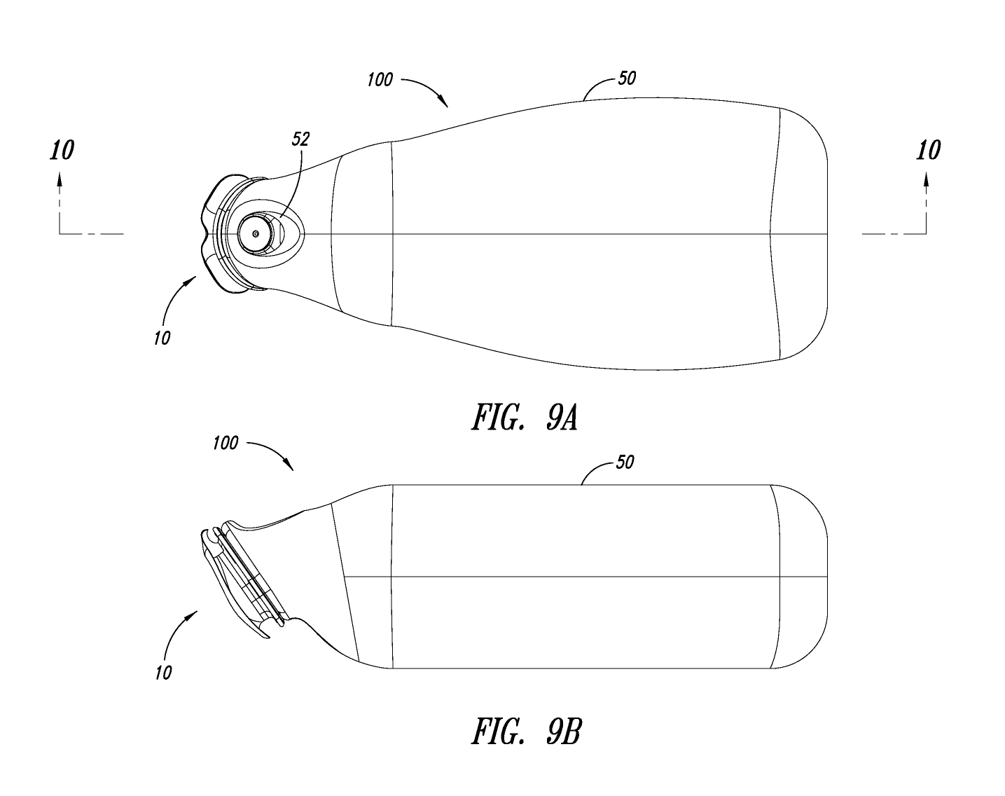

FIGS. 1-11 illustrate an integrated assembly 100 according to one example. FIG. 1 is an isometric view of the integrated assembly 100. The integrated assembly in this view includes a mask subassembly 10, an aroma subassembly 20, an articulated neck portion 30, and a base subassembly 40. The base subassembly 40 defines an air inlet 42 at one end thereof. FIGS. 8-10 further illustrate a thermal cover subassembly 50 in combination with the other elements of the integrated assembly 100.

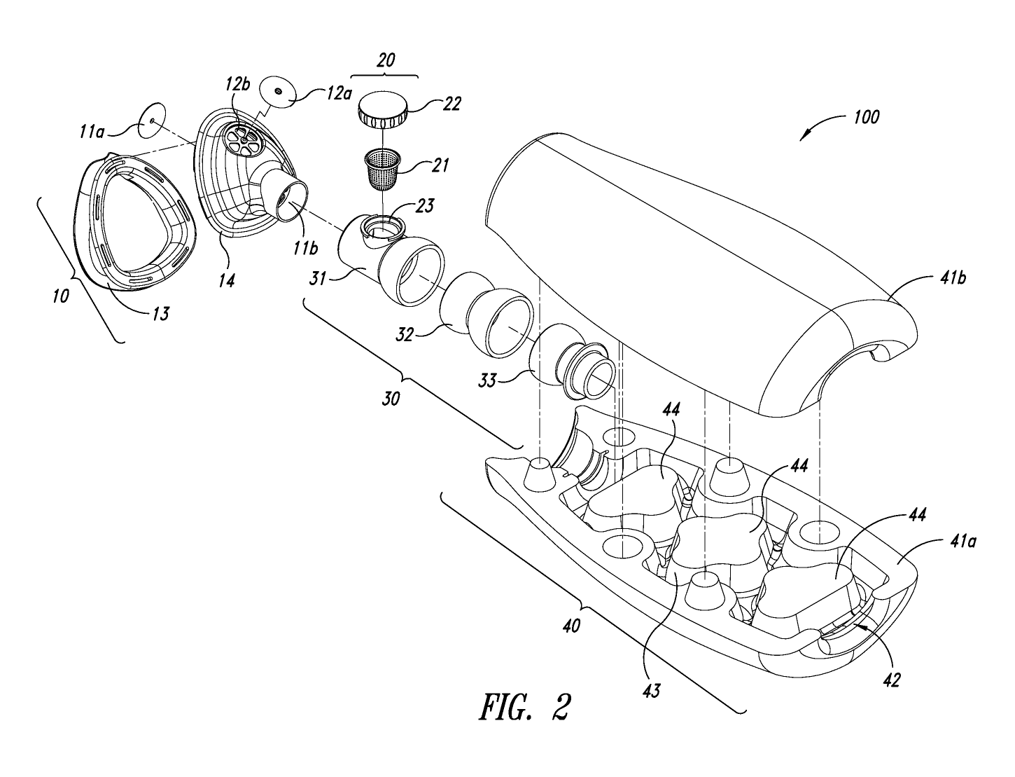

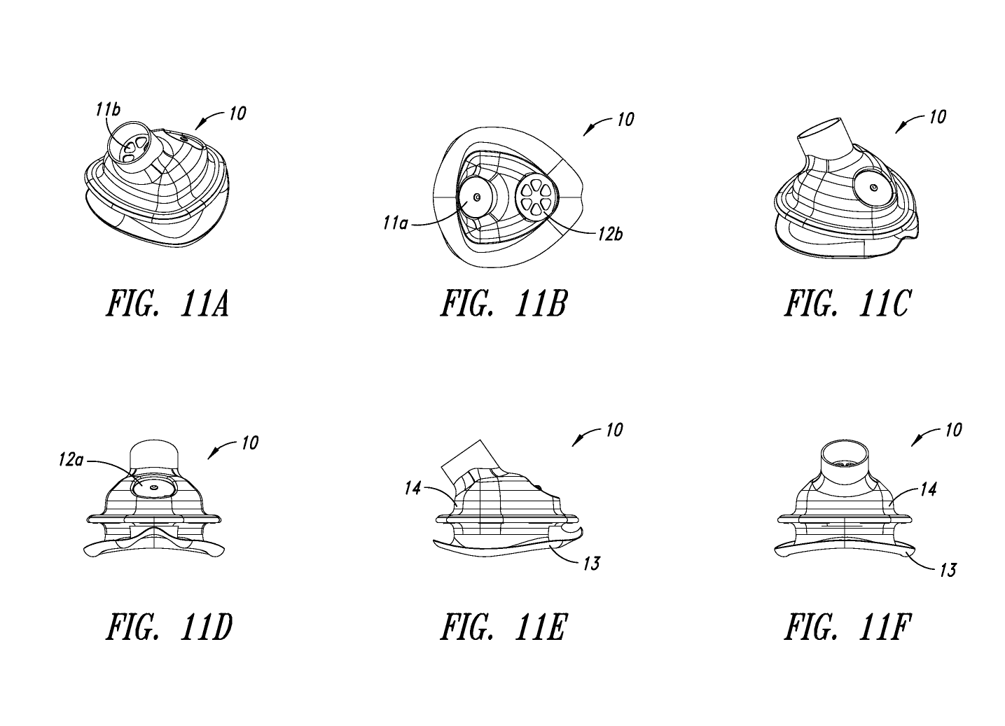

FIG. 2 is an exploded, isometric view of the integrated assembly 100. The mask subassembly 10 includes a mask cushion 13 and a mask frame 14 that supports the mask subassembly 10. FIGS. 11a-11f include further views of the mask subassembly 10. The mask frame can be, for example, a rigid plastic frame made of materials Tritan, ABS, and/or nylon, for example. The mask cushion 13 can be a soft rubber mask cushion that is flexible and shaped to fit around and seal with a broad spectrum of faces and nose types. Materials for the mask cushion 13 may include silicone and/or urethane.

The mask subassembly 10 can further include an inlet opening 11b and a one-way flapper valve 11a positioned at the entry of the neck to the mask to enable air flow to be inhaled into the mask. The mask subassembly 10 can also include an outlet opening 12b and a one-way flapper valve 12a positioned on the top of the mask frame 14 to enable air flow to be exhaled out of the mask. Both flapper valves can be made of flexible material such as silicone or urethane. One purpose of the flapper valves is to reduce air thermal convection losses.

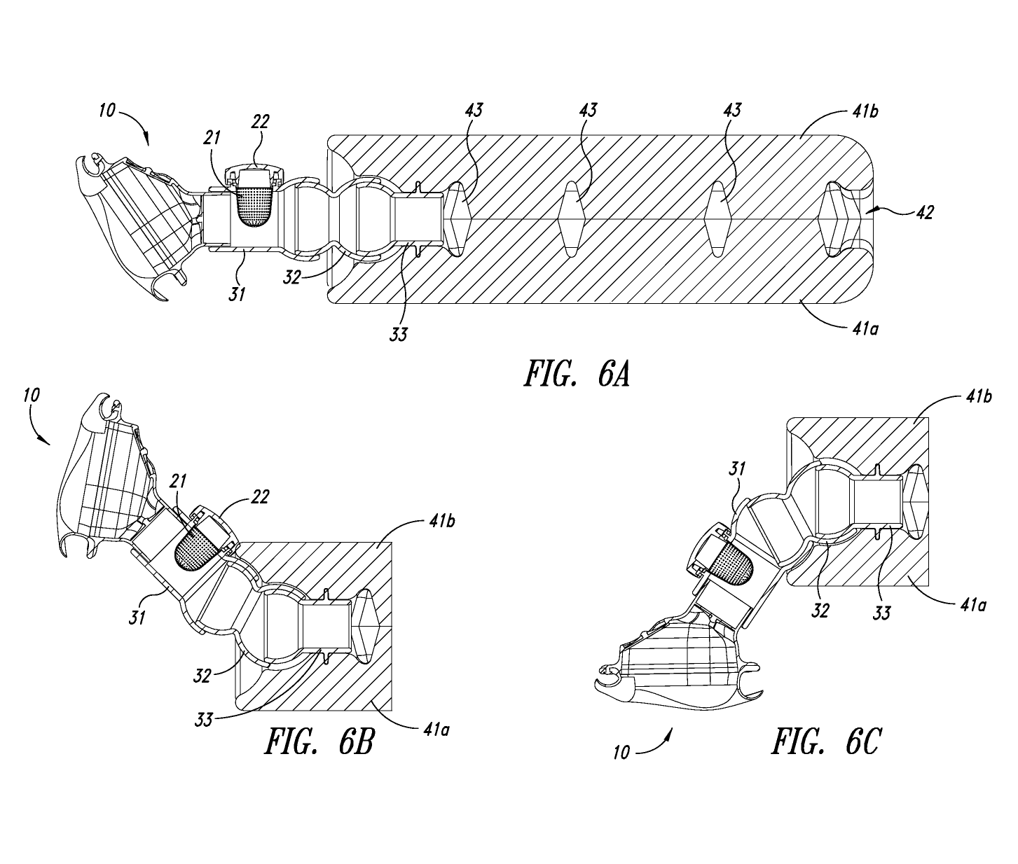

The neck portion 30 of the illustrated integrated assembly 100 includes three segments 31, 32, and 33 of rigid plastic to enable bending into a plurality of positions. Materials for the neck portion 30 may include Tritan, ABS, and/or nylon. The neck portion 30 can be securely positioned straight, as shown in FIG. 6a or it may securely bend up, as shown in FIG. 6b, or bend down for comfortable mask positioning, as shown in FIG. 6c.

The neck segment 31 includes an opening 23 for insertion of the aroma subassembly 20. The aroma subassembly 20 includes a rigid plastic cup 21 and removable rigid plastic cap 22 that is easily opened and securely closed. Some examples can include a flexible plastic tether to keep the cap 22 from being lost when opened. Materials for the rigid plastic material include ABS, PP, LDPE, and Tritan. Materials for the flexible plastic tether may include urethane. The neck portion 30 in combination with the shape of the rigid plastic frame 14 of the mask subassembly 10 and the flexure and shape of the rubber mask cushion 13 ensure a secure fit and seal around the nose for a broad spectrum of faces and nose types.

In this example, the bottom of the cup 21 is a perforated or mesh plastic or stainless mesh that sets securely into a cutout in the neck of the mask subassembly 20 such that the perforated or mesh portion of the cup 21 of the aroma subassembly 21 is inside the neck segment 31 of the neck portion 30. This enables the air flow through the aroma subassembly to be infused with aromatic phytochemicals from aromatic material that may be inserted into the cup 21 by the user. Thermally-conditioned air flows through the wire mesh or perforated aroma subassembly to pick up phytochemical scents.

The cap 22 may be removed to fill the aroma subassembly with aromatic oil, herbs, or flowers that infuse the passing air with aromatic phytochemicals in all positions of the neck portion 30. For example, air flow passes through the aroma subassembly whether the neck portion 30 is in straight position (FIG. 6a), or bent up (FIG. 6b) or bent down (FIG. 6c).

The base subassembly 40 of the integrated assembly 100 includes a rigid plastic frame and a thermal mass. Materials for the thermal mass may include ceramic, porcelain, stoneware, terracotta, or gel pack materials. The base assembly 40 of this example has a clamshell construction with “bosses” for securing assembly of bottom half 41a to top half 41b when in use. The thermal cover 50, described below, also assists in securing assembly when in use. The bottom half 41a and the top half 41b each include channels 43 defined by lands 44. With this construction, ambient air enters the base subassembly through the air inlet 42 at the bottom of the base subassembly 40. Air is inhaled through the channels 43 in the thermal mass, being cooled, warmed, or maintained at ambient temperature as it passes. Thermally-conditioned air then enters the neck portion 30.

Each of the components discussed above can be, in some examples, top shelf dishwasher-safe.

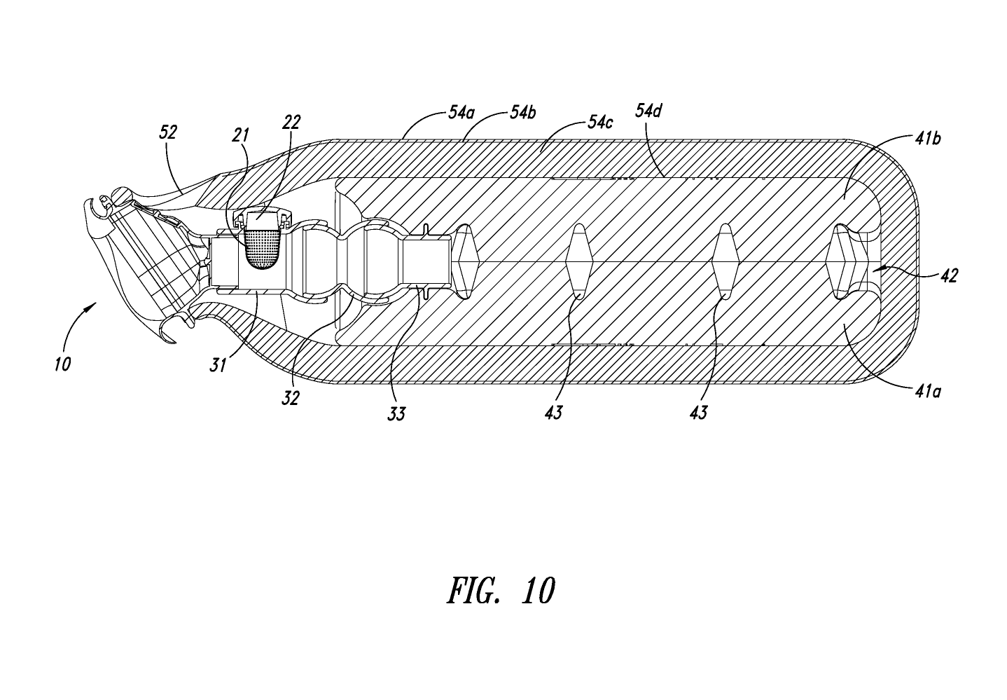

FIGS. 8-10 further illustrate a thermal cover subassembly 50 in combination with the other elements of the integrated assembly 100. The integrated assembly 100 can be enclosed during use with the thermal cover 50 to maintain the coolth, warmth, or ambient temperature of the thermal mass at an optimal temperature. The thermal cover 50 attaches to the mask 10 to keep the mask and neck insulated. As shown in FIG. 10, the thermal cover can be layered with a plush outer fabric layer 54a, a heat reflector layer 54b, insulation layer 54c, and lining fabric layer 54d. The layers may be made of a plurality of textures and materials that will be insulative and also may offer wicking properties to condensate, such as synthetic fleece, hemp, and/or poly fill fabrics. The heat reflector layer 54b of the thermal cover 50 is lined with heat reflective material such as an aluminized Mylar. The size and color of the thermal cover 50 will be a factor in determining the degree to which the thermal cover insulates the thermal mass.

In addition to a tensioning band, the thermal cover 50 also functions to hold the two clamshell halves 41a and 41b of the base subassembly 40 securely together during use.

The heat-reflective lining layer 54b, insulation layer 54c, and lining fabric layer 54d of the thermal cover each has an opening at its base for the base subassembly air inlet. The thermal cover exterior fabric acts as an air filter at 51 (FIG. 8) for inhaled air through the base subassembly 40 air inlet 42.

There is a through-hole 52 to the thermal cover 50 to vent the one-way flapper valve 12a in the mask subassembly 10. The thermal cover 50 has an opening at the neck of the mask subassembly for the soft rubber mask cushion 13 of the protrusion of the mask subassembly 10.

The soft exterior layer 54a of the thermal cover 50 can enhance the effectiveness of the methods discussed below by permitting comfortable and easy “cradling” of the integrated assembly. The thermal cover can be launderable.

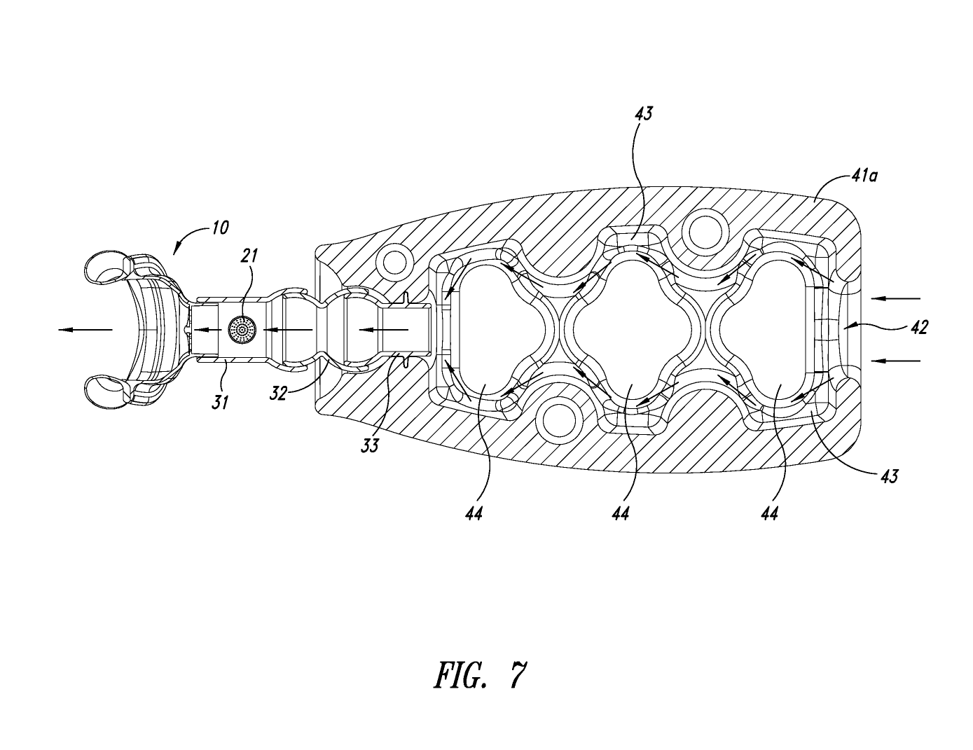

FIG. 7 illustrates how air flows through the integrated assembly 100 during use. Ambient air enters the integrated assembly 100 through the air inlet 42 at the bottom of the base subassembly 40. Air is inhaled through the channels 43 in the thermal mass, being cooled, warmed, or maintained at ambient temperature as it passes. Thermally-conditioned air enters the neck portion 30. Thermally-conditioned air flows through the wire mesh or perforated cup 21 of the aroma subassembly 20 to pick up phytochemical scents. Air flow passes through the aroma subassembly 20 whether the neck portion 30 is in straight position (FIG. 6a), or bent up (FIG. 6b) or bent down (FIG. 6c). Air flows through the one-way flapper valve 11a and into the upper portion of the mask subassembly 10.

Cooled, warmed, or ambient temperature air and/or aromatically conditioned air is then inhaled through the nose. Such inhaled air may also be inhaled through the mouth to more rapidly initiate facilitation of air flow at the onset of use.

Air is exhaled then through the mouth. Alternatively, exhaled, warmed air may flow through the second one-way flapper valve 12a and out of the mask subassembly 10, limiting the formation of condensate and helping to maintain an optimal temperature in the subassemblies.

In other examples, the shape of the mask and mask cushion are customized for use in positions that primarily enable inhalations and exhalations from the mouth.

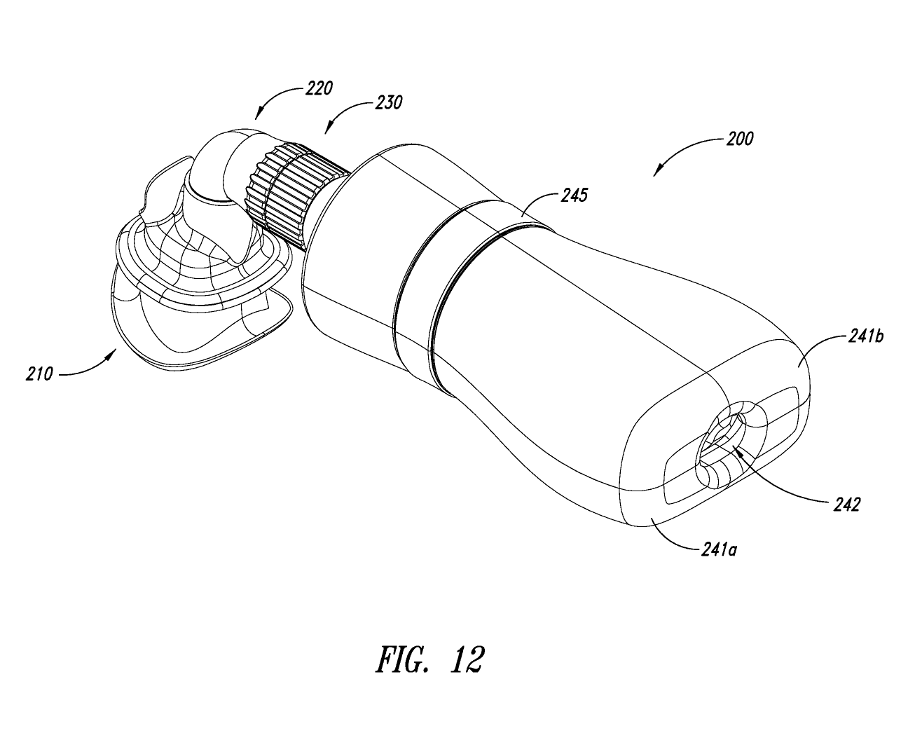

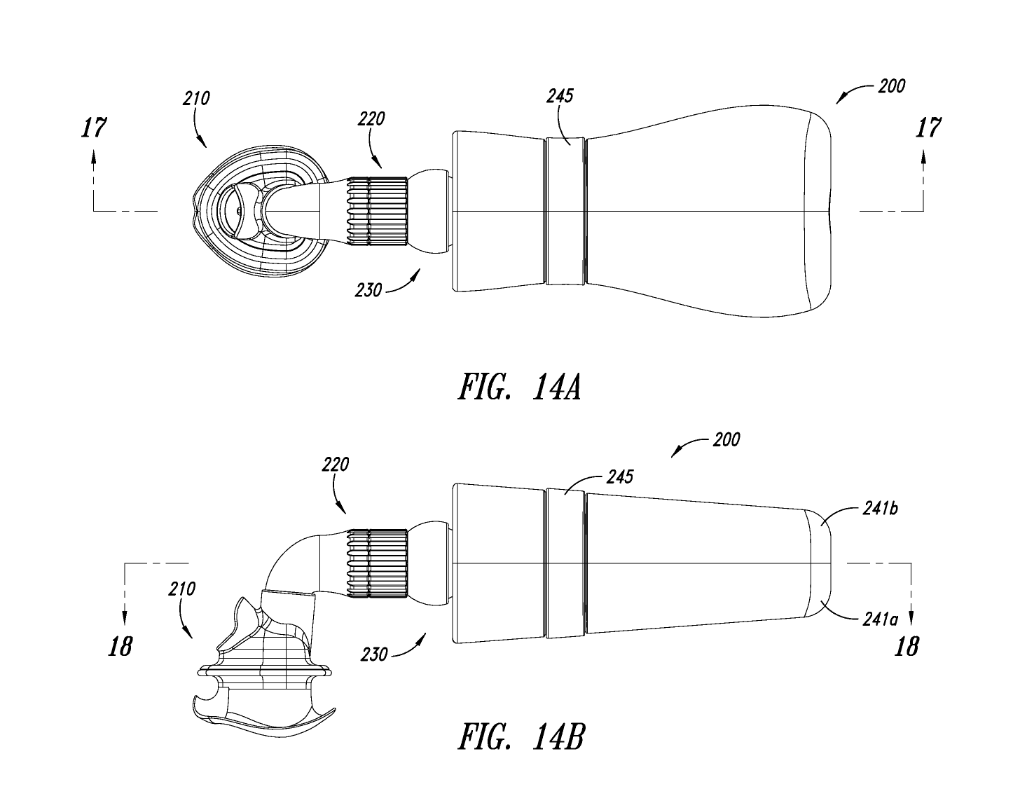

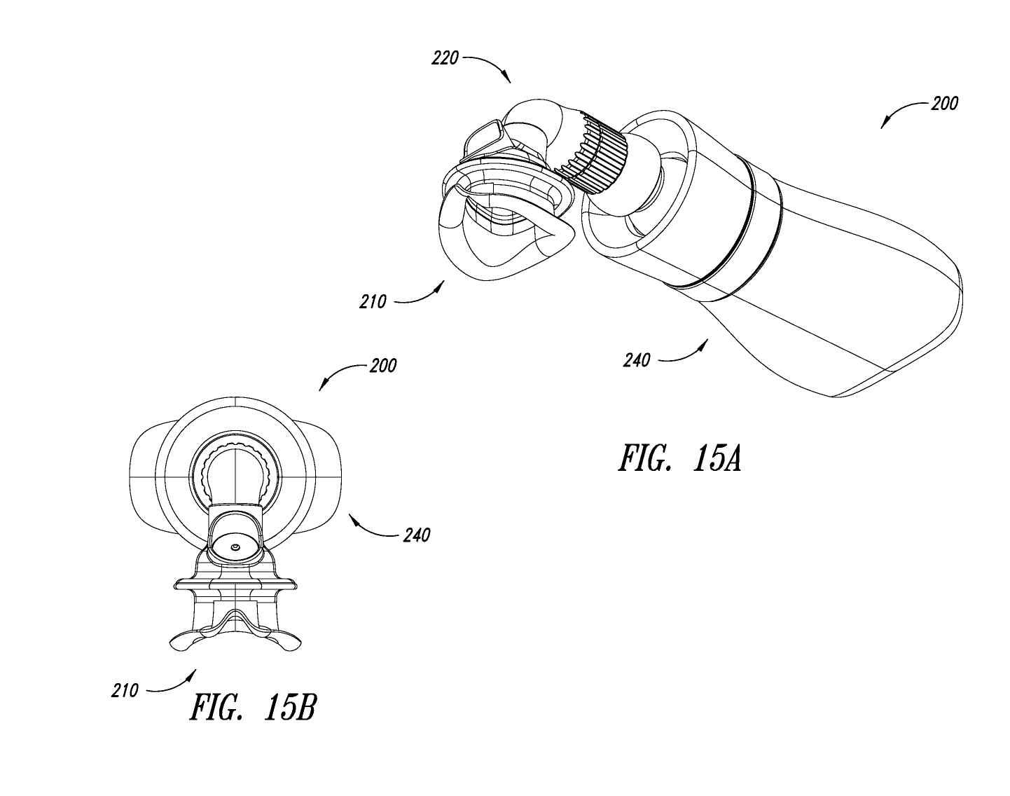

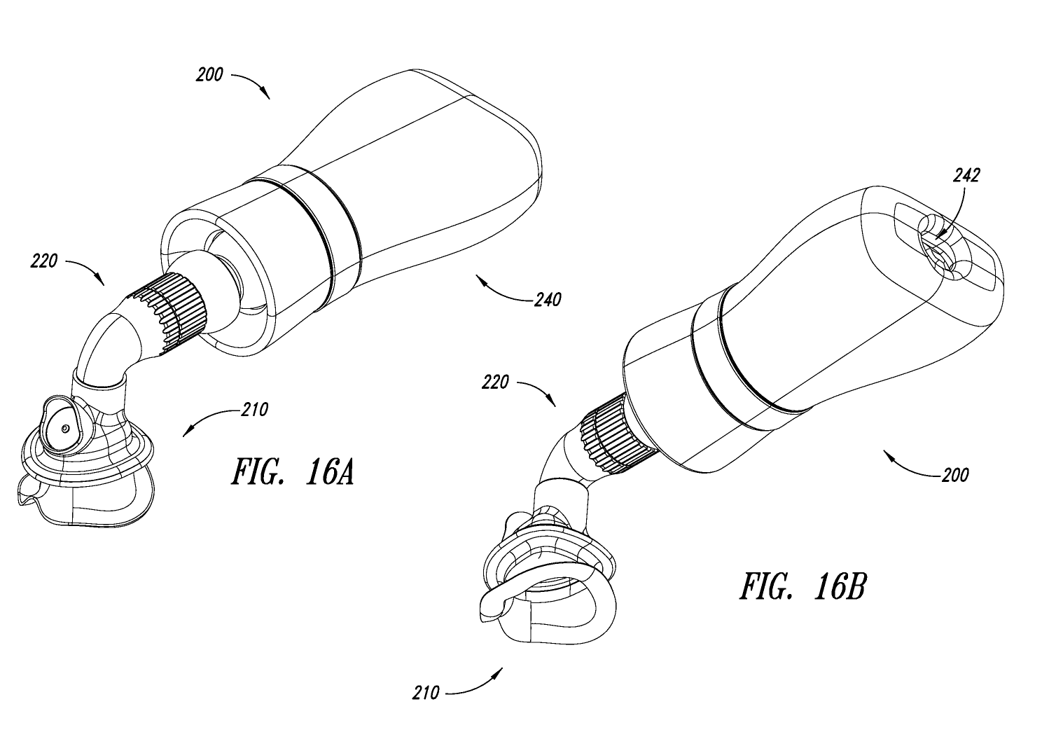

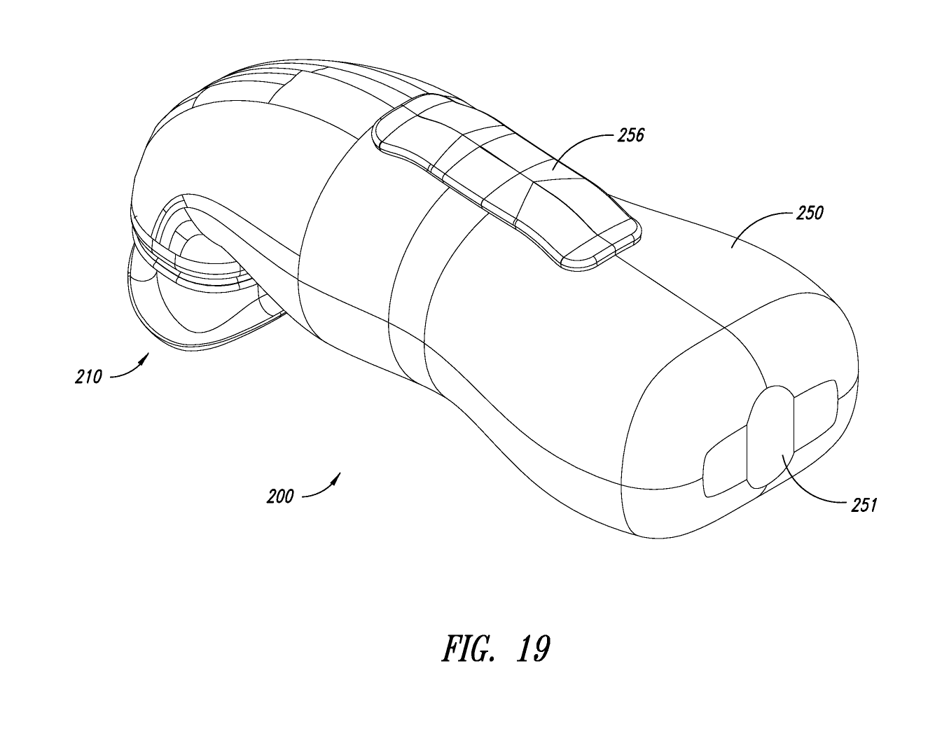

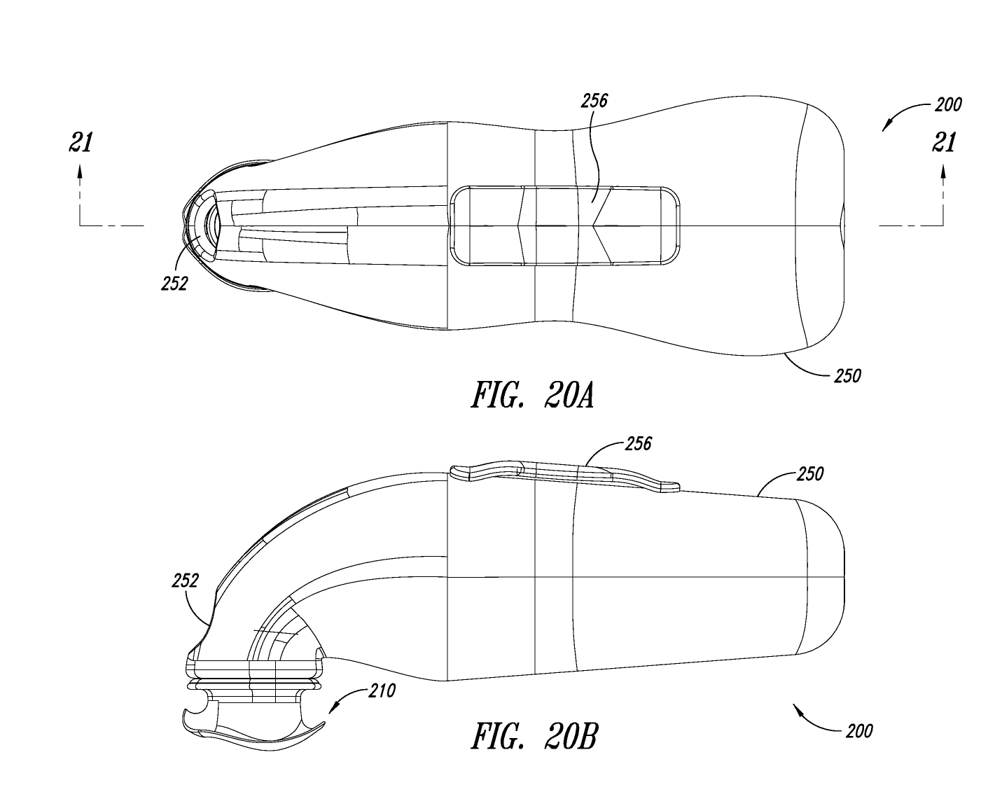

FIGS. 12-23 illustrate an integrated assembly 200 according to another aspect of the present disclosure. FIG. 12 is an isometric view of the integrated assembly 200. The integrated assembly in this view includes a mask subassembly 210, an aroma subassembly 220, an articulated neck portion 230, and a base subassembly 240 including lower and upper clam shell portions 241a and 241b secured together by a thermal mass band 245. The base subassembly 240 defines an air inlet 242 at one end thereof. FIGS. 19-21 further illustrate a thermal cover subassembly 250 in combination with the other elements of the integrated assembly 200.

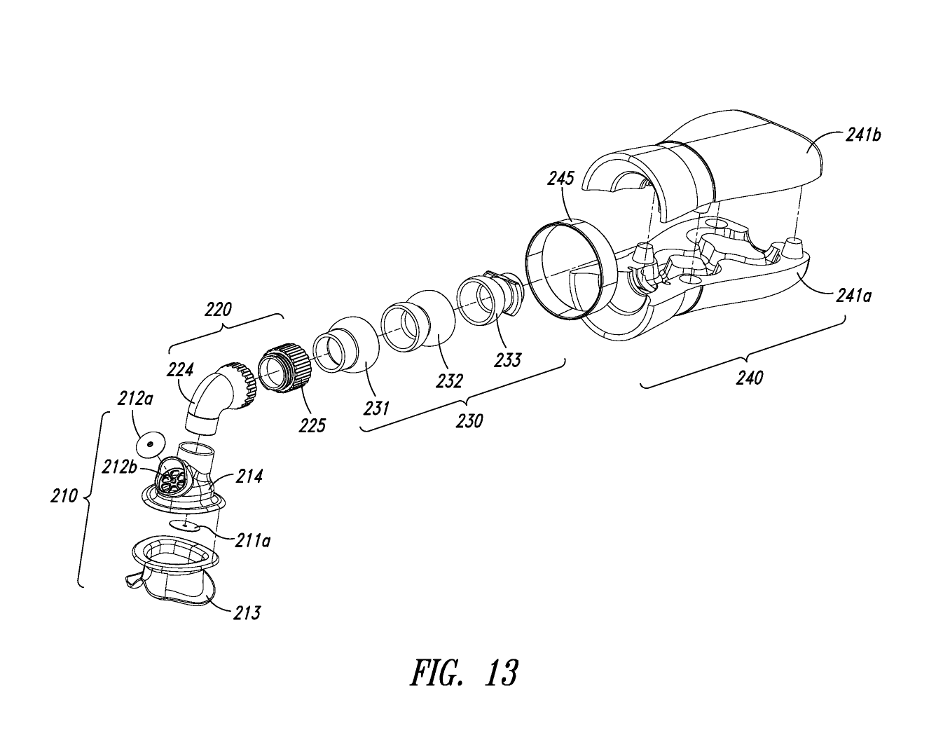

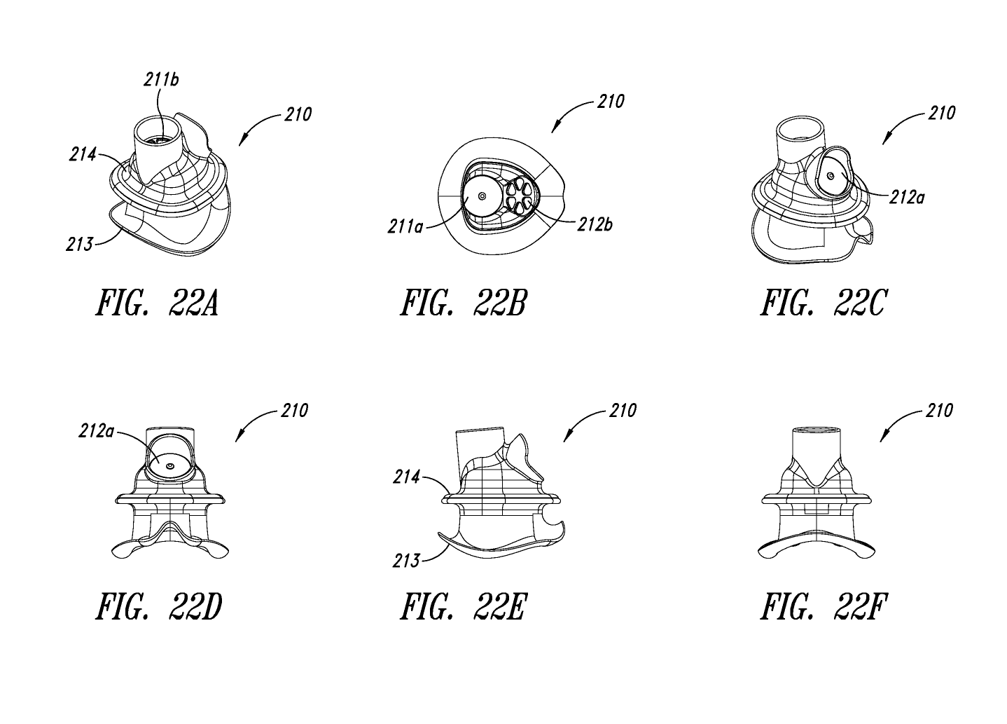

FIG. 13 is an exploded, isometric view of the integrated assembly 200. The mask subassembly 210 includes a mask cushion 213 and a mask frame 214 that supports the mask subassembly 210. FIGS. 22a-22f include further views of the mask subassembly 210. The mask frame 214 can be, for example, a rigid plastic frame made of materials Tritan, ABS, and/or nylon, for example. The mask cushion 213 can be a soft rubber mask cushion that is flexible and shaped to fit around and seal with a broad spectrum of faces and nose types. Materials for the mask cushion 213 may include silicone and/or urethane.

The mask subassembly 210 can further include an inlet opening 211b and a one-way flapper valve 211a positioned at the entry of the neck to the mask to enable air flow to be inhaled into the mask. The mask subassembly 210 can also include an outlet opening 212b and a one-way flapper valve 212a positioned on the top of the mask frame 214 to enable air flow to be exhaled out of the mask. Both flapper valves can be made of flexible material such as silicone or urethane. One purpose of the flapper valves is to reduce air thermal convection losses.

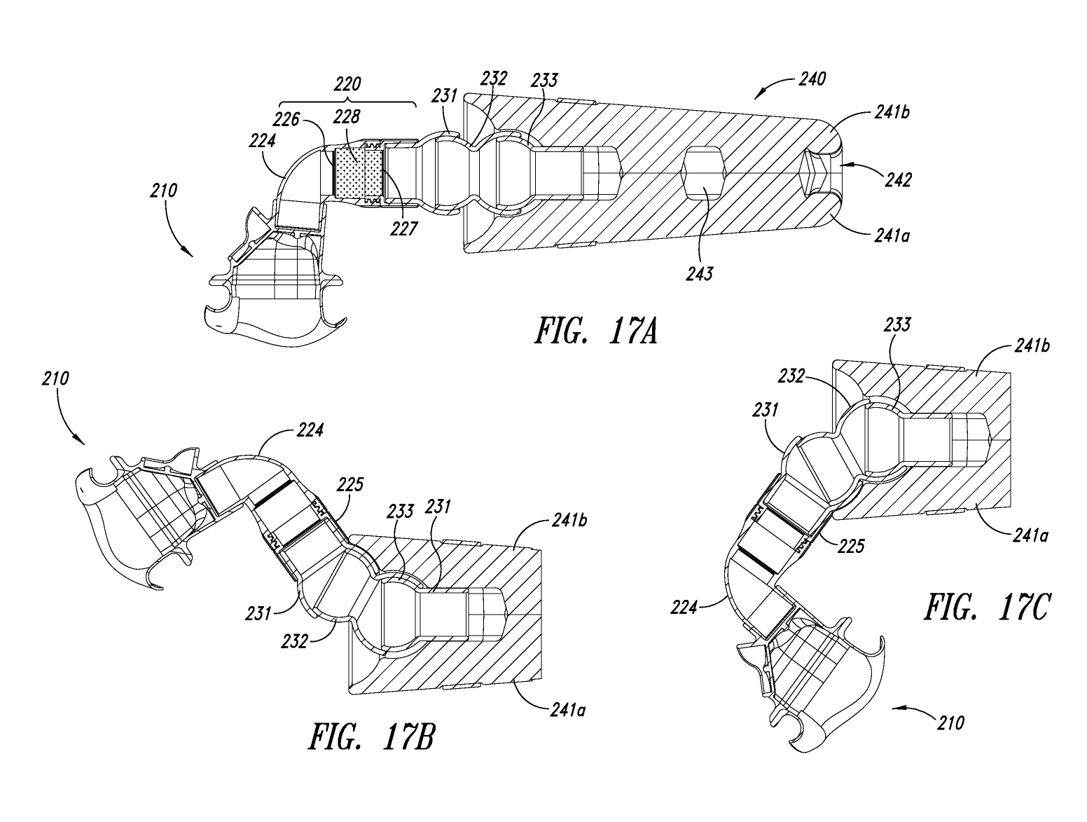

The neck portion 230 of the illustrated integrated assembly 100 includes three segments 231, 232, and 233 of rigid plastic to enable bending into a plurality of positions. Materials for the neck portion 230 may include Tritan, ABS, and/or nylon. The neck portion 230 can be securely positioned straight, as shown in FIG. 17a or it may securely bend up, as shown in FIG. 17b, or bend down for comfortable mask positioning, as shown in FIG. 17c.

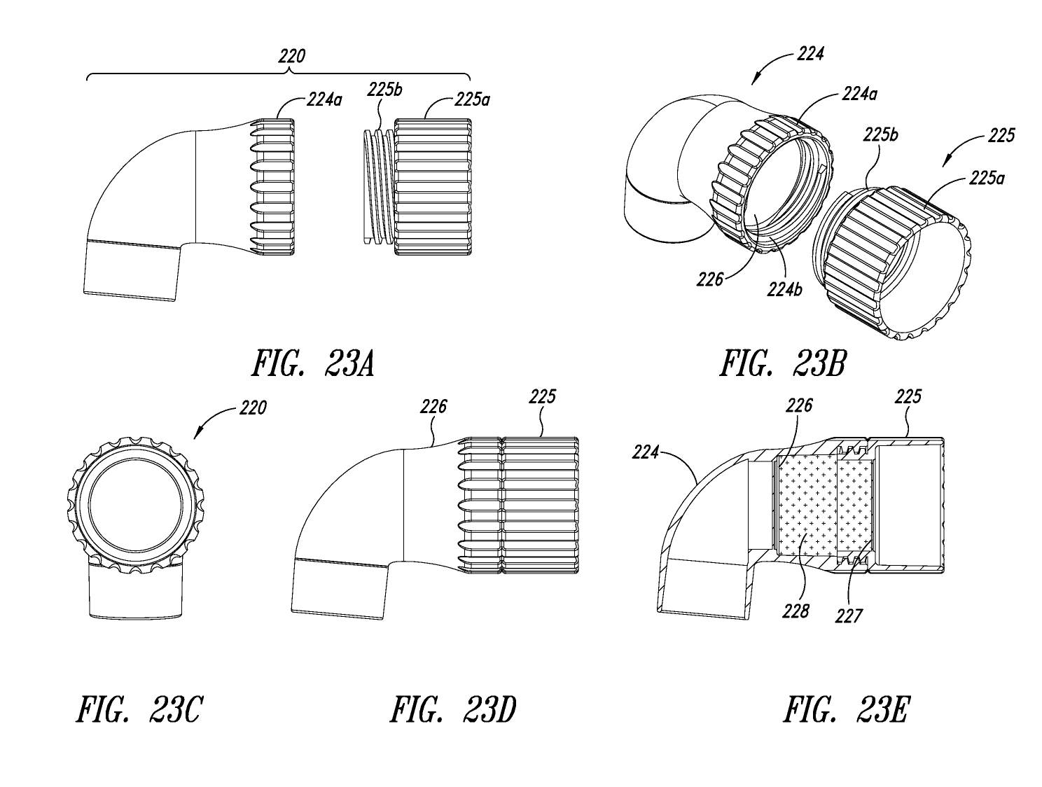

The neck segment 231 is coupled to the aroma subassembly 320. As best shown in FIGS. 23a-23e, the aroma subassembly 320 includes an aroma chamber elbow 224 and an aroma chamber cap 225. The aroma chamber elbow 224 and the aroma chamber cap 225 include mating threads 224b and 225b, respectively, so that these separate halves can be separated or joined. Knurled surface 224a and 225b can aid in the screwing and unscrewing. The aroma chamber elbow 224 and the aroma chamber cap 225 respectively include screens 226 and 227, which may be plastic, that define an aroma chamber 228 when the aroma chamber elbow 224 and the aroma chamber cap 225 are joined.

By unscrewing the aroma chamber elbow 224 and the aroma chamber cap 225, the aroma chamber 228 can be filled with aromatic oil, herbs, or flowers that infuse the passing air with aromatic phytochemicals in all positions of the neck portion 230. For example, air flow passes through the aroma subassembly 220 whether the neck portion 230 is in straight position (FIG. 17a), or bent up (FIG. 17b) or bent down (FIG. 17c).

The base subassembly 240 of the integrated assembly 200 can include a rigid plastic frame and a thermal mass. Materials for the thermal mass may include ceramic, porcelain, stoneware, terracotta, or gel pack materials. The base assembly 240 of this example has a clamshell construction with “bosses” for securing assembly of bottom half 241a to top half 241b when in use with the aid of the thermal mass band 245. The thermal mass band 245 can be made of, for example, flexible silicon rubber. The thermal cover 250, described below, can also assists in securing assembly when in use. The bottom half 241a and the top half 241b each include channels 243 defined by lands 244. With this construction, ambient air enters the base subassembly through the air inlet 242 at the bottom of the base subassembly 240. Air is inhaled through the channels 243 in the thermal mass, being cooled, warmed, or maintained at ambient temperature as it passes. Thermally-conditioned air then enters the neck portion 230.

Each of the components discussed above can be, in some examples, top shelf dishwasher-safe.

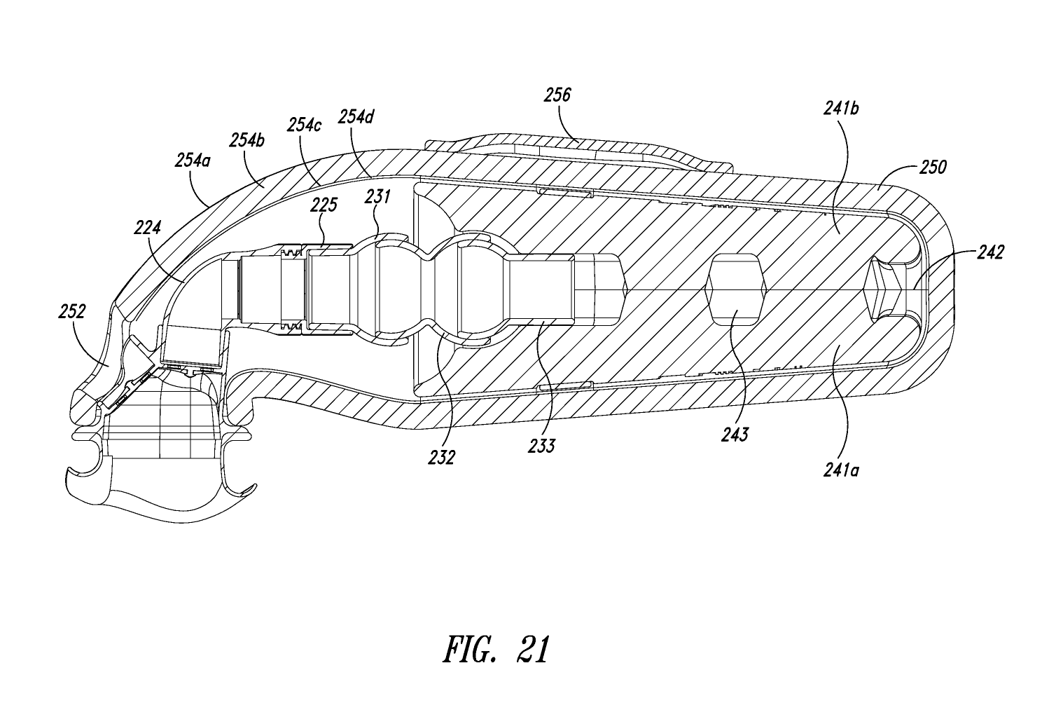

FIGS. 19-21 further illustrate a thermal cover subassembly 250 in combination with the other elements of the integrated assembly 200. The integrated assembly 200 can be enclosed during use with the thermal cover 250 to maintain the coolth, warmth, or ambient temperature of the thermal mass at an optimal temperature. The thermal cover 250 attaches to the mask 210 to keep the mask and neck insulated. The thermal cover 250 can also include a handle 256 to aid in holding the integrated assembly 200 during use of for carrying the integrated assembly 200. As shown in FIG. 21, the thermal cover 250 can be layered with a plush outer fabric layer 254a, a heat reflector layer 254b, insulation layer 254c, and lining fabric layer 254d. The layers may be made of a plurality of textures and materials that will be insulative and also may offer wicking properties to condensate, such as synthetic fleece, hemp, and/or poly fill fabrics. The heat reflector layer 254b of the thermal cover 250 is lined with heat reflective material such as an aluminized Mylar. The size and color of the thermal cover 250 will be a factor in determining the degree to which the thermal cover insulates the thermal mass.

In addition to a tensioning band, the thermal cover 250 also functions to hold the two clamshell halves 241a and 241b of the base subassembly 240 securely together during use.

The heat-reflective lining layer 254b, insulation layer 254c, and lining fabric layer 254d of the thermal cover each has an opening at its base for the base subassembly air inlet. The thermal cover exterior fabric acts as an air filter at 251 (FIG. 19) for inhaled air through the base subassembly 240 air inlet 242.

There is a through-hole 252 to the thermal cover 250 to vent the one-way flapper valve 212a in the mask subassembly 210. The thermal cover 250 has an opening at the neck of the mask subassembly for the soft rubber mask cushion 213 of the protrusion of the mask subassembly 210.

The soft exterior layer 254a of the thermal cover 250 can enhance the effectiveness of the methods discussed below by permitting comfortable and easy “cradling” of the integrated assembly. The thermal cover can be launderable.

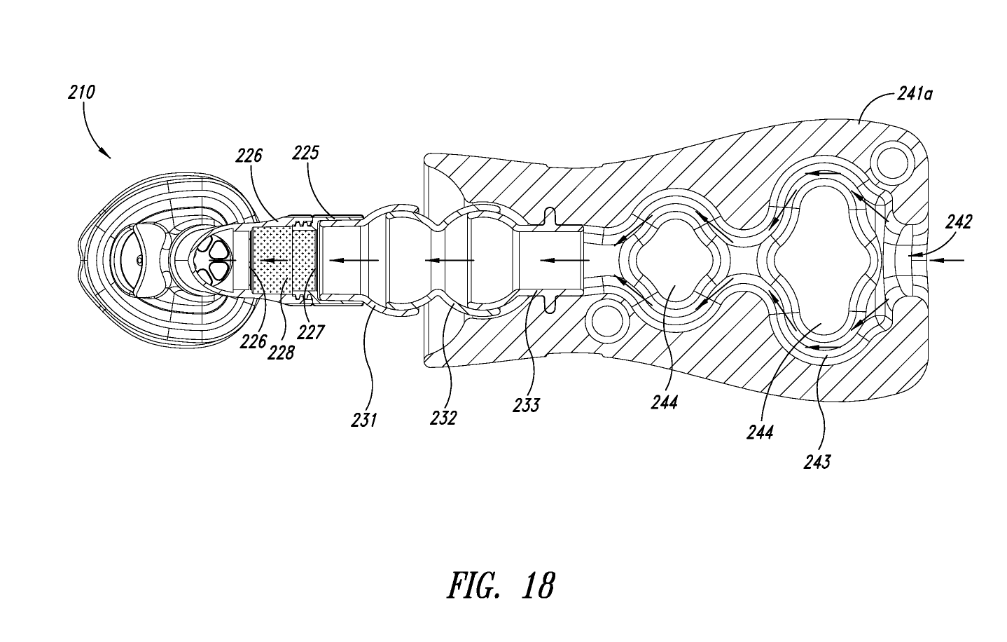

FIG. 18 illustrates how air flows through the integrated assembly 200 during use. Ambient air enters the integrated assembly 200 through the air inlet 242 at the bottom of the base subassembly 240. Air is inhaled through the channels 243 in the thermal mass, being cooled, warmed, or maintained at ambient temperature as it passes. Thermally-conditioned air enters the neck portion 230. Thermally-conditioned air flows through the aroma subassembly 220 to pick up phytochemical scents. Air flow passes through the aroma subassembly 220 whether the neck portion 230 is in straight position (FIG. 17a), or bent up (FIG. 17b) or bent down (FIG. 17c). Air flows through the one-way flapper valve 211a and into the upper portion of the mask subassembly 210.

Cooled, warmed, or ambient temperature air and/or aromatically conditioned air is then inhaled through the nose. Such inhaled air may also be inhaled through the mouth to more rapidly initiate facilitation of air flow at the onset of use.

Air is exhaled then through the mouth. Alternatively, exhaled, warmed air may flow through the second one-way flapper valve 212a and out of the mask subassembly 210, limiting the formation of condensate and helping to maintain an optimal temperature in the subassemblies.

In other examples, the shape of the mask and mask cushion are customized for use in positions that primarily enable inhalations and exhalations from the mouth.

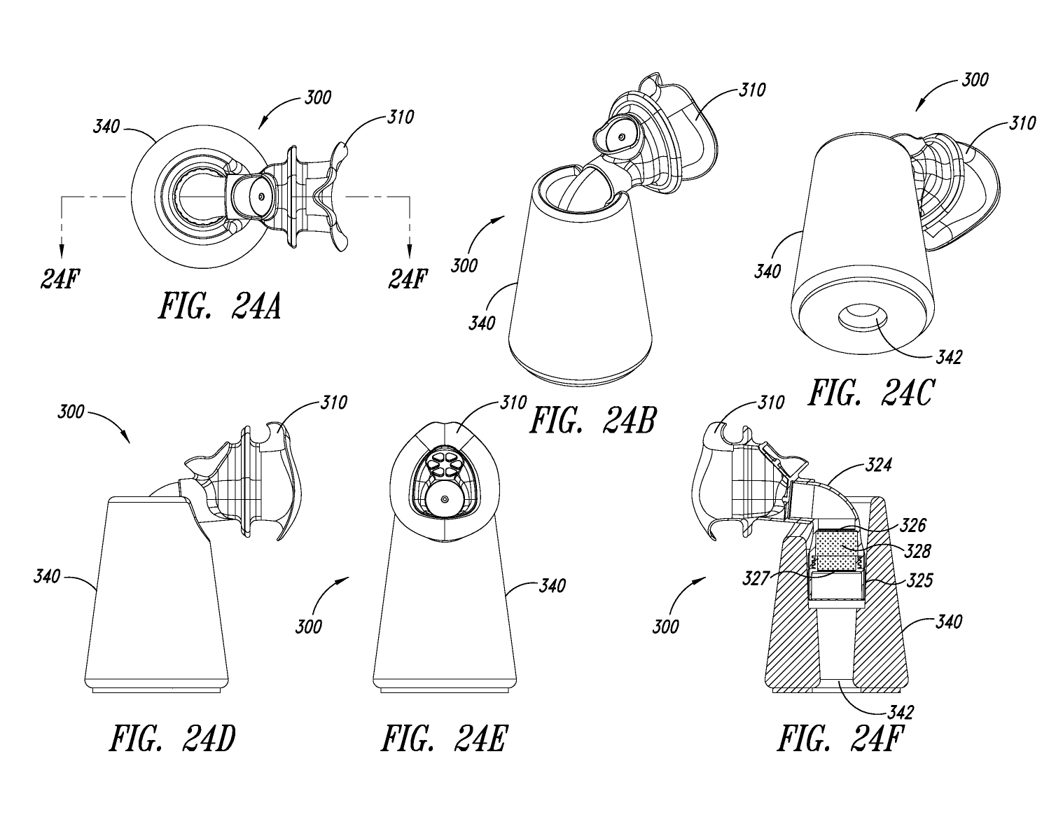

FIGS. 24a-24f illustrate an integrated assembly 300 according to another aspect of the present disclosure. The integrated assembly 300 is a mini unit that can use a mask 310 and aroma chamber components that are the same as the mask 210 and aroma subassembly 220 described above. For example, the aroma chamber elbow 324, aroma chamber cap 325, screens 326 and 327, and aroma chamber 328 can be similar or identical to the corresponding components of the aroma subassembly 220 described above. The thermal mass 340 includes an inlet 342 and is shaped to stand up on a table. In this example, there is no adjustable neck portion.

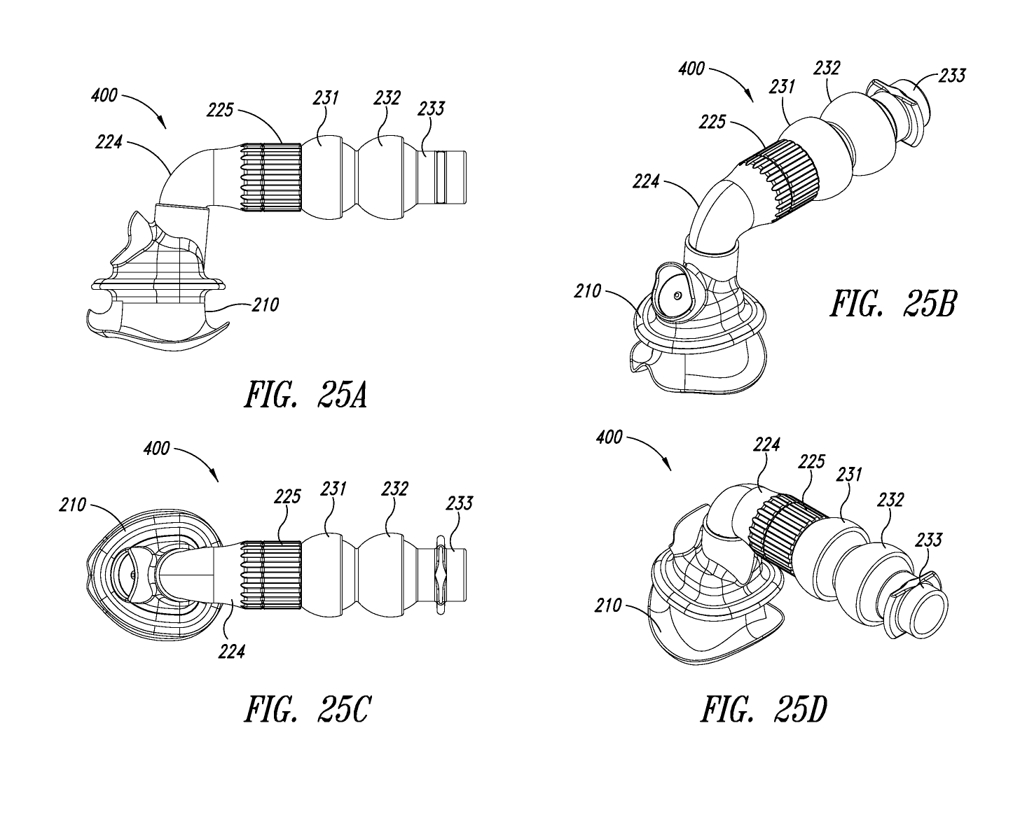

FIGS. 25a-25d illustrate an integrated assembly 400 according to another aspect of the present disclosure. This example includes the same components of the mask 210, aroma chamber 220, and neck portion 230 as described above. However, in this example, there is no base subassembly 240. Instead, this unit operates with air at ambient temperatures. The neck portion 230 can act as a handle.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including but not limited to U.S. Provisional Patent Application Ser. No. 61/650,279 and U.S. Provisional Patent Application Ser. No. 61/734,868, are incorporated herein by reference, in their entirety. Aspects of the embodiments can be modified, if necessary, to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A device to enable user inhalation and exhalation of aromatically conditioned air, comprising:
a mask subassembly; and
an aroma subassembly positioned relative to the mask subassembly such that air drawn through the first opening passes through the aroma subassembly prior to exiting through the mask.

2. A device to enable user inhalation and exhalation of ambient air and/or thermally conditioned air comprising:
a mask subassembly;
a neck portion including a first opening on one end and a second opening on an opposite end thereof with a passage extending between the first and second openings, the passage of the neck portion in fluid communication with the mask subassembly such that air drawn through the first opening travels through the neck portion and exits through the mask; and
a thermal mass that includes an inlet and a channel, the channel in fluid communication with the mask subassembly such that air that enters the thermal mass through the inlet is cooled, warmed, or maintained at ambient temperature before it reaches the mask subassembly.

3. A device to enable user inhalation and exhalation of aromatically conditioned and/or ambient air and/or thermally conditioned air comprising:
a mask subassembly;
an aroma subassembly positioned relative to the mask subassembly and the thermal mass such that air drawn through the inlet passes through the aroma subassembly prior to exiting through the mask;
a neck portion including a first opening on one end and a second opening on an opposite end thereof with a passage extending between the first and second openings, the passage of the neck portion in fluid communication with the mask subassembly such that air drawn through the first opening travels through the neck portion and exits through the mask;
a thermal mass that includes an inlet and a channel, the channel in fluid communication with the mask subassembly such that air that enters the thermal mass through the inlet is cooled, warmed, or maintained at ambient temperature before it reaches the mask subassembly; and
a cover subassembly.

4. The device of claim 1, wherein the mask subassembly includes an inlet opening and a first one-way flapper valve positioned at an entry to the mask subassembly to enable air flow to be inhaled into the mask subassembly.

5. The device of claim 4, wherein the mask subassembly includes an outlet opening and a second one-way flapper valve positioned on the outlet opening of the mask subassembly to enable air flow to be exhaled out of the mask.

6. The device of claim 1, wherein the aroma subassembly includes an in-line aroma chamber.

7. The device of claim 6, wherein the in-line aroma subassembly elbow and connector respectively include screens that define an aroma chamber when the aroma chamber elbow and the aroma chamber cap are joined.

8. The device of claim 2, wherein the neck portion includes a plurality of articulable segments.

9. The device of claim 8, wherein the plurality of articulable segments are arranged such that air drawn through the first opening passes through the aroma subassembly prior to exiting through the mask regardless of a relative orientation of the plurality of segments.

10. The device of claim 3, wherein the thermal mass includes an inlet and a channel, the channel in fluid communication with the passage of the neck portion such that air that enters the thermal mass through the inlet is cooled, warmed, or maintained at ambient temperature before it reaches the passage of the neck portion.

11. The device of claim 2, wherein the thermal mass includes two clam-shell portions or gel packs that, when combined, define the channel.

12. The device of claim 2, further comprising a cover that at least substantially surrounds the thermal mass.

13. The device of claim 12, wherein the cover attaches to the mask subassembly to keep the mask subassembly and neck portion insulated.

14. The device of claim 12, wherein the cover includes a plurality of layers.

15. The device of claim 14, wherein the plurality of layers includes an outer fabric layer, a heat reflector layer, an insulation layer, and lining fabric layer.

16. The device of claim 15 wherein the heat-reflective lining layer, insulation layer, and lining fabric layer of the cover each includes an opening corresponding to the inlet of the thermal mass, and the outer fabric layer acts as an air filter for inhaled air through the inlet of the thermal mass.

17. A method of enabling user inhalation and exhalation of air and/or self-administering aromatically conditioned air, and/or ambient air and/or thermally conditioned air, the method comprising the steps of:
providing a device to enable user inhalation and exhalation of air and/or aromatically conditioned and/or ambient air and/or thermally conditioned air, the device comprising:
a mask subassembly;
an aroma subassembly;
a neck portion;
a thermal mass that includes an inlet and a channel, the channel in fluid communication with the mask subassembly; and
a cover subassembly; and
drawing air through the mask subassembly such that ambient air enters the inlet, is inhaled through the channels, and then flows into the mask subassembly.

18. The method of claim 17, further comprising inhaling the aromatically conditioned and/or ambient air and/or thermally conditioned air into the nose via the mask subassembly.

19. The method of claim 17, further comprising exhaling the aromatically conditioned and/or ambient air and/or thermally conditioned air from the nose into the mask subassembly.

20. The method of claim 17, further comprising exhaling the aromatically conditioned and/or ambient air and/or thermally conditioned air from the mouth.

21. The method of claim 17, further comprising using the device to teach breathing techniques conducive to relaxation, sleep, alertness, meditation, mindful breathing, and/or other user-selected breathing techniques using sensory feedback from the device, including aroma and/or temperature and/or sound and/or tactile sensations from air flow.

22. The method of claim 17, further comprising using the device to establish and heighten awareness of positive sleep routines and rituals, such as use of the device, that are considered important in healthy sleep hygiene.

23. The method of claim 17, further comprising using the device, with check lists, schedules, or calendars that include preparation, use, and care of the device, to establish good sleep hygiene habits and identify behaviors that are not conducive to good sleep hygiene.

24. The method of claim 17, further comprising using the device with journal formats or logs, that include prompting questions about use of the device, that are designed to assist the user in identifying dysfunctional rumination and belief systems about sleep and relaxation.

25. The method of claim 17, comprising using the device with a plurality of user-selected cognitive and/or behavioral modification means to induce relaxation, sleep, and/or alertness, which may incorporate self-administered or professionally administered instructions in the use of the device and/or forms for written exercises to be used specifically in connection with the device such as i) journal forms, ii) check lists, iii) calendars, and/or iv) schedules.

26. A method of enabling user inhalation and exhalation of air, the method comprising the steps of:
providing a device to enable user inhalation of self-selected ambient air and exhalation of user-warmed air, the device comprising:
a mask subassembly; and
drawing air through the mask subassembly such that ambient air enters the inlet, is inhaled through the channels, flows into the mask subassembly; and is exhaled.

27. The method of claim 26, further comprising inhaling air into the nose via the mask subassembly.

28. The method of claim 26, further comprising exhaling air from the nose into the mask subassembly.

29. The method of claim 26, further comprising inhaling air into the mouth via the mask subassembly.

30. The method of claim 26, further comprising exhaling air from the mouth.

31. The method of claim 26, further comprising use of the device to teach breathing techniques conducive to relaxation, sleep, alertness, meditation, mindful breathing, and/or other user-selected breathing techniques using sensory feedback from the device, including temperature and/or sound and/or tactile sensations from air flow.

32. The method of claim 26, further comprising use of the device to establish and heighten awareness of positive sleep routines and rituals, such as use of the device, that are considered important in healthy sleep hygiene.

33. The method of claim 26, further comprising use of the device, with check lists, schedules, or calendars that include preparation, use, and care of the device, to establish good sleep hygiene habits and identify behaviors that are not conducive to good sleep hygiene.

34. The method of claim 26, further comprising use of the device with journal formats or logs, that include prompting questions about use of the device, that are designed to assist the user in identifying dysfunctional rumination and belief systems about sleep and relaxation.

35. A method of claim 26, comprising use of the device with a plurality of user-selected cognitive and/or behavioral modification means to induce relaxation, sleep, and/or alertness, which may incorporate self-administered or professionally administered instructions in the use of the device and/or forms for written exercises to be used specifically in connection with the device such as i) journal forms, ii) check lists, iii) calendars, and/or iv) schedules.
